(19) United States
(12) Patent Application Publication
(10) Pub. No.: US 2024/0197661 A1
(43) Pub. Date: Jun. 20, 2024

(54) ORAL DOSAGE FORMS OF METHYL HYDROGEN FUMARATE AND PRODRUGS THEREOF

(71) Applicant: Arbor Pharmaceuticals, LLC, Atlanta, GA (US)

(72) Inventors: Peter A. VIRSIK, Portola Valley, CA (US); Sami KARABORNI, Cupertino, CA (US); Laura Elizabeth BAUER, Sunnyvale, CA (US); Chen MAO, Mountain View, CA (US); Ching Wah CHONG, Fremont, CA (US)

(21) Appl. No.: 18/492,882

(22) Filed: Oct. 24, 2023

Related U.S. Application Data
(63) Continuation of application No. 16/566,696, filed on Sep. 10, 2019, now abandoned.
Application No. 16/566,696 is a continuation-in-part of application No. 13/973,622, filed on Aug. 22, 2013, now abandoned.
(60) Provisional application No. 61/692,168, filed on Aug. 22, 2012.
(60) Provisional application No. 61/692,174, filed on Aug. 22, 2012.
(60) Provisional application No. 61/692,179, filed on Aug. 22, 2012.
(60) Provisional application No. 61/713,897, filed on Oct. 15, 2012.
(60) Provisional application No. 61/713,961, filed on Oct. 15, 2012.
(60) Provisional application No. 61/733,234, filed on Dec. 4, 2012.
(60) Provisional application No. 61/769,513, filed on Feb. 26, 2013.
(60) Provisional application No. 61/837,796, filed on Jun. 21, 2013.
(60) Provisional application No. 61/841,513, filed on Jul. 1, 2013.

(51) Int. Cl. A61K 31/197; A61K 9/20; A61K 9/28; A61P 17/06

(52) U.S. Cl. CPC A61K 31/197; A61K 9/2009; A61K 9/2013; A61K 9/2018; A61K 9/2866; A61P 17/06

(57) ABSTRACT

Methods of treating psoriasis with sustained release compression coated tablet dosage forms comprising certain MHF prodrugs are provided.

This application is a continuation-in-part of U.S. Non-Provisional Application Ser. No. 13/973,622, filed Aug. 22, 2013, which claims priority to U.S. Provisional Application Ser. Nos. 61/692, 179 filed Aug. 22, 2012, 61/692,168, filed Aug. 22, 2012, 61/713,897 filed Oct. 15, 2012, 61/733,234 filed Dec. 4, 2012, 61/769,513 filed Feb. 26, 2013, 61/841,513 filed Jul. 1, 2013, 61/692,174 filed Aug. 22, 2012, and 61/713,961 filed Oct. 15, 2012, 61/837,796 filed Jun. 21, 2013 the contents of each of which are incorporated by reference in their entirety.

### TECHNICAL FIELD

The present disclosure relates to oral dosage forms of methyl hydrogen fumarate (MHF) and prodrugs of MHF which are useful in treating conditions such as multiple sclerosis (MS) and/or psoriasis.

### BACKGROUND

Fumaric acid esters, i.e., dimethylfumarate (DMF) in combination with salts of ethylhydrogenfumarate, have been used in the treatment of psoriasis for many years. The combination product, marketed under the trade name Fumaderm®, is in the form of oral tablets and is available in two different dosage strengths (Fumaderm® initial and Fumaderm®):

| | Fumaderm ® | Fumaderm ® |
|---|---|---|
| Fumarate Compound | Initial (mg) | (mg) |
| Dimethylfumarate | 30 | 120 |
| Ethyl hydrogen fumarate, | 67 | 87 |
| calcium salt | | |
| Ethyl hydrogen fumarate, | 5 | 5 |
| magnesium salt | | |
| Ethyl hydrogen fumarate, | 3 | 3 |
| zinc salt | | |

The two strengths are intended to be applied in an individually based dosing regimen starting with Fumaderm® initial in an escalating dose, and then after, e.g., three weeks of treatment, switching to Fumaderm®. Both Fumaderm® initial and Fumaderm® are enteric coated tablets.

Another marketed composition is Fumaraat 120® containing 120 mg of DMF and 95 mg of calcium monoethyl fumarate (TioFarma, Oud-Beijerland, Netherlands). The pharmacokinetic profile of Fumaraat 120® in healthy subjects is described in Litjens et al., Br. J. Clin. Pharmacol., 2004, vol. 58:4, pp. 429-432. The results show that a single oral dose of Fumaraat 120° is followed by a rise in serum MHF concentration and only negligible concentrations of DMF and fumaric acid is observed. Thus, DMF is thought to be a precursor or prodrug of MHF.

U.S. Pat. Nos. 6,277,882 and 6,355,676 disclose respectively the use of alkyl hydrogen fumarates and the use of certain fumaric acid monoalkyl ester salts for preparing microtablets for treating psoriasis, psoriatic arthritis, neurodermatitis and enteritis regionalis Crohn. U.S. U.S. Pat. No. 6,509,376 discloses the use of certain dialkyl fumarates for the preparation of pharmaceutical preparations for use in transplantation medicine or the therapy of autoimmune diseases in the form of microtablets or micropellets. U.S. Pat. No. 4,959,389 discloses compositions containing different salts of fumaric acid monoalkyl esters alone or in combination with a dialkyl fumarate. GB 1,153,927 relates to medical compositions comprising dimethyl maleic anhydride, dimethyl maleate and/or DMF.

Biogen Idec's BG12, an oral dosage form of DMF that is an enteric coated capsule containing DMF in micropellet form, has been in human clinical testing for the treatment of MS and has shown promising results in reducing MS relapses and MS disability progression. Unfortunately, DMF is highly irritating to the skin and mucosal membranes with the result that oral administration of DMF tends to cause serious digestive tract irritation with attendant nausea, vomiting, abdominal pain and diarrhea. This irritation problem is particularly problematic with the mucosal tissue lining the stomach. For this reason, products such as Fumaderm® and BG12 are made with enteric coatings that prevent the DMF from being released from the dosage form until after the dosage form passes out of the stomach and into the small intestine.

More recently, MHF prodrugs including (N,N-Diethylcarbamoyl)methyl methyl (2E)but-2-ene-1,4-dioate and (N,N-Dimethylcarbamoyl)methyl methyl (2E)but-2-ene-1,4-dioate are disclosed in Gangakhedkar et al. U.S. Pat. No. 8,148,414. Additional MHF prodrugs are disclosed in Cundy et al. U.S. Patent Application 61/595,835 filed Feb. 7, 2012. Both of these disclose the use of MHF prodrugs for treating a number of medical conditions, including MS and psoriasis.

### SUMMARY

Disclosed herein are orally administered compression coated tablet dosage forms of methyl hydrogen fumarate, or a prodrug of methyl hydrogen fumarate, having improved prodrug stability and shelf-life. The dosage forms are useful for treating conditions such as multiple sclerosis and psoriasis.

Fumaric acid esters such as methyl hydrogen fumarate and prodrugs of methyl hydrogen fumarate, e.g., dimethyl fumarate, have certain physical and chemical properties that cause problems when such compounds are used as therapeutic agents, particularly when administered orally to a patient. First, such compounds have been shown to cause skin irritation. Second, such compounds exhibit degrees of chemical instability upon exposure to light, including ultra violet light. Third, such compounds have been shown to cause flushing in certain patients and/or at certain dosages. Fourth, certain fumarate compounds (i.e., dimethyl fumarate) have been shown to cause adverse interactions with the endothelial tissues lining the stomach, causing severe tissue damage and attendant gastrointestinal distress and symptoms such as nausea and abdominal pain and diarrhea. Fifth, such compounds tend to be chemically less stable at low PH levels (e.g., pH≤2), compared to nearer neutral pH levels (e.g., pH of 3 to 6) with the result that the compounds can chemically break down into non-therapeutic metabolites in the low pH environs of the stomach. While enteric coatings have previously been proposed for certain fumarate dosage forms, it has now been discovered that these fumarate compounds tend to exhibit poor chemical stability in the presence of such enteric coating materials.

These and other problems are solved by an oral pharmaceutical tablet comprising a tablet core and a compressed coating layer surrounding the tablet core. The tablet core contains a compound selected from (i) methyl hydrogen fumarate (MHF), (ii) a prodrug of MHF, pharmaceutically acceptable salts thereof and combinations thereof, and (iii) one or more core tableting excipients, such as a binder, a filler, a glidant and/or a lubricant. The compressed coating layer comprises a material that is either (i) a proton-donating acidic material having a pKa of greater than 8, (ii) a proton-accepting basic material having a pKa of less than 2, (iii) a natural gum or polysaccharide, (iv) a neutral polymer salt, (v) a sugar, or (vi) a lipid. The coating layer also remains intact and releases no more than 20% of the compound over a period of 2 hours after the tablet is placed in an aqueous solution free of the compound.

In certain embodiments, the coating layer material is a non-ionizable polymer substantially free of carboxylic acid moieties. In particular, the coating layer material may be selected from non-ionizable cellulosic polymers, non-ionizable vinyl polymers, and non-ionizable polyvinyl alcohol polymers. The coating layer optionally includes one or more excipients selected from binders, fillers, glidants and lubricants.

In certain embodiments, at least one of the tablet core and the coating layer comprises a sustained release agent. In particular, the sustained release agent may be selected from hydroxypropyl methyl cellulose and ethyl cellulose.

In certain embodiments, the tablet has a core weight to compressed coating weight ratio of 1:1 to 1:3. In other embodiments, the tablet releases no more than 10% of the compound over a period of 2 hours after the tablet is placed in an aqueous solution free of the compound.

In certain embodiments, the compressed coating layer comprises a material that is either (i) a proton-donating acidic material having a pKa of greater than 10, or (ii) a proton-accepting basic material having a pKa of less than 0.

In certain embodiments, the compound comprises methyl hydrogen fumarate. In other embodiments, the compound comprises a prodrug of methyl hydrogen fumarate. In still other embodiments, the prodrug of methyl hydrogen fumarate is selected from dimethyl fumarate, (N,N-Diethylcarbamoyl)methyl methyl (2E)but-2-ene-1,4-dioate, (N,N-Dimethylcarbamoyl)methyl methyl (2E)but-2-ene-1,4-dioate, and combinations thereof.

In certain embodiments, the tablet core is a an immediate release formulation and the compression coated tablet releases at least 80% of the compound within 3 hours after being placed in an aqueous solution free of the compound. In other embodiments, the tablet core is a sustained release formulation and the compression coated tablet releases at least 80% of the compound over a period of at least 6 hours after being placed in an aqueous solution free of the compound.

Also provided are methods of treating a disease in a patient, comprising orally administering to a patient in need thereof the pharmaceutical tablets disclosed herein. In particular, the tablets disclosed herein can be used to treat multiple sclerosis and/or psoriasis.

### BRIEF DESCRIPTION OF THE DRAWINGS

FIG. 1 is a graph showing the in vitro MHF prodrug release profile (percent MHF prodrug released over time) for the dosage forms of Example 1, tested in accordance with Example 4;

FIG. 2 is a graph showing the in vitro MHF prodrug release profile (percent MHF prodrug released over time) for the dosage forms of Example 2, tested in accordance with Example 4;

FIG. 3 is a graph showing the in vitro MHF prodrug release profile (percent MHF prodrug released over time) for the dosage forms of Example 3, tested in accordance with Example 4;

FIG. 4 is a graph showing the concentration of MHF in the blood of fasted monkeys following administration of the oral dosage forms of Examples 1 and 2;

FIG. 5 is a graph showing the concentration of MHF in the blood of fed monkeys following administration of the oral dosage forms of Examples 1 and 2;

FIG. 6 is a graph showing the in vitro MHF prodrug release profile (percent MHF prodrug released over time) for the dosage forms as well as the uncoated cores of Example 3, tested in accordance with Example 6;

FIG. 7 is a graph showing the in vitro MHF prodrug release profile (percent MHF prodrug released over time) for the dosage forms as well as the uncoated cores of Example 2, tested in accordance with Example 7;

FIG. 8 is a graph showing the in vitro MHF prodrug release profile (percent MHF prodrug released over time) for the dosage forms of Examples 1 and 8, tested in accordance with Example 8;

FIG. 9 is a graph showing the in vitro MHF prodrug release profile (percent MHF prodrug released over time) for the dosage forms of Example 9, tested in accordance with Example 9;

FIG. 10 is a graph showing the in vitro MHF prodrug release profile (percent MHF prodrug released over time) for the dosage forms of Examples 8 and 10, tested in accordance with Example 10;

FIG. 11 is a graph showing the in vitro MHF prodrug release profile (percent MHF prodrug released over time) for the dosage forms of Example 11, tested in accordance with Example 11;

FIG. 12 is a graph showing the in vitro MHF prodrug release profile (percent MHF prodrug released over time) for the dosage forms of Example 12, tested in accordance with Example 12;

FIG. 13 is a graph showing the concentration of MMF in the blood in fed and fasted healthy human patients following administration of the oral dosage form of Example 3;

FIG. 14 is a graph showing the rate of degradation of DMF and (N,N-Diethylcarbamoyl) methyl methyl (2E)but-2-ene-1,4-dioate as a function of increased acetate concentration;

FIG. 15 is a graph showing the rate of formation of degradation products for (N,N-Diethylcarbamoyl)methyl methyl (2E)but-2-ene-1,4-dioate as tested in Example 14;

FIG. 16 is a graph showing the in vitro MHF prodrug release profile (percent MHF prodrug released over time) for the dosage forms of Examples 15-18, tested in accordance with Example 21;

FIG. 17 is a graph showing the in vitro MHF prodrug release profile (percent MHF prodrug released over time) for the dosage forms of Example 19, tested in accordance with Example 22;

FIG. 18 is a graph showing the in vitro MHF prodrug release profile (percent MHF prodrug released over time) for the dosage forms of Example 20, tested in accordance with Example 23;

FIG. 19 is a graph showing the in vitro MHF prodrug release profile (percent MHF prodrug released over time) for the dosage forms of Example 26, tested in accordance with Example 28; and

FIG. 20 is a graph showing the in vitro MHF prodrug release profile (percent MHF prodrug released over time) for the dosage forms of Example 27, tested in accordance with Example 29.

### DEFINITIONS

A dash (“-”) that is not between two letters or symbols is used to indicate a point of attachment for a moiety or substituent. For example, —CON H₂ is bonded through the carbon atom.

“Alkyl” refers to a saturated or unsaturated, branched, cyclic, or straight-chain, monovalent hydrocarbon radical derived by the removal of one hydrogen atom from a single carbon atom of a parent alkane, alkene, or alkyne. Examples of alkyl groups include, for example, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-I-yl, prop-2-yn-1-yl, etc.; butyls such as butan-1-yl, butan-2-yl, 2-methyl-propan-I-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, cyclopropyl, cyclobutyl, cyclopentyl, etc.; and the like.

The term “alkyl” includes groups having any degree or level of saturation, i.e., groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl, or alkynyl are used. The term “alkyl” includes cycloalkyl and cycloalkylalkyl groups. In certain embodiments, an alkyl group can have from 1 to 10 carbon atoms (C₁₋₁₀), in certain embodiments, from 1 to 6 carbon atoms (C₁₋₆), in certain embodiments from 1 to 4 carbon atoms (C₁₋₄), in certain embodiments, from 1 to 3 carbon atoms (C₁₋₃), and in certain embodiments, from 1 to 2 carbon atoms (C₁₋₂). In certain embodiments, alkyl is methyl, in certain embodiments, ethyl, and in certain embodiments, n-propyl or isopropyl.

“Arylalkyl” refers to an acyclic alkyl radical in which one of the hydrogen atoms bonded to a carbon atom, typically a terminal or spa carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C_{8-20,} in certain embodiments, an arylalkyl group is C₈₋₁₈ arylalkyl, e.g., the alkanyl, alkenyl or alkynyl moiety of the arylalkyl group is C₁₋₈ and the aryl moiety is C_{6-10.} In certain embodiments, an arylalkyl group is C₇₋₁₂ arylalkyl.

“AUC” refers to the area under a curve on which time is plotted on the X-axis and concentration of a substance (e.g., MHF) in blood or blood plasma is plotted on the Y-axis over a particular period of time (e.g., time zero to 24 hours). AUC is commonly expressed in units of mg-hr/ml.

“Compounds” include MHF and MHF prodrugs. MHF products include DMF and the compounds of Formula (I) or Formula (II) including any specific compounds within these formulae. Compounds may be identified either by their chemical structure and/or chemical name. Compounds are named using Chemistry 4-D Draw Pro, version 7.01c (ChemInnovation Software, Inc., San Diego, CA). When the chemical structure and chemical name conflict, the chemical structure is determinative of the identity of the compound. The compounds described herein may comprise one or more chiral centers and/or double bonds and therefore may exist as stereoisomers such as double bond isomers (i.e., geometric isomers), enantiomers, or diastereomers. Accordingly, any chemical structures within the scope of the specification depicted, in whole or in part, with a relative configuration are deemed to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well-known to those skilled in the art. Compounds of Formula (I) or Formula (II) include, for example, optical isomers of compounds of Formula (I) or Formula (II), racemates thereof, and other mixtures thereof. In such embodiments, a single enantiomer or diastereomer, i.e., optically active form, can be obtained by asymmetric synthesis or by resolution of the racemates. Resolution of the racemates may be accomplished, for example, by methods such as crystallization in the presence of a resolving agent, or chromatography using, for example, chiral stationary phases. Notwithstanding the foregoing, in compounds of Formula (I) or Formula (II) the configuration of the illustrated double bond is only in the E configuration (i.e., trans configuration).

“Compressed coating layer” refers to the coating layer of a tablet-in-tablet composition, which is produced by first preparing a tablet “core” from a first component, and then applying a coating layer by a subsequent compression step. The terms “shell” and “mantle” are also sometimes used to describe the compressed coating layer.

MHF and MHF prodrug compounds also include isotopically labeled compounds where one or more atoms have an atomic mass different from the atomic mass conventionally found in nature. Examples of isotopes that may be incorporated into the compounds disclosed herein include, for example, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N oxides. In general, compounds disclosed herein may be free acid, hydrated, solvated, or N oxides. Certain compounds may exist in multiple crystalline, co-crystalline, or amorphous forms. Compounds of Formula (I) or Formula (II) include pharmaceutically acceptable salts thereof or pharmaceutically acceptable solvates of the free acid form of any of the foregoing, as well as crystalline forms of any of the foregoing.

MHF and MHF prodrug compounds also include solvates. A solvate refers to a molecular complex of a compound with one or more solvent molecules in a stoichiometric or non-stoichiometric amount. Such solvent molecules include those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules are water.

Further, when partial structures of the compounds are illustrated, an asterisk (0*) indicates the point of attachment of the partial structure to the rest of the molecule.

“Cycloalkyl” refers to a saturated or partially unsaturated cyclic alkyl radical. Where a specific level of saturation is intended, the nomenclature cycloalkanyl or cycloalkenyl is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₃₋₈ cycloalkyl.

“Cycloalkylalkyl” refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀, and in certain embodiments, a cycloalkylalkyl group is C₃₋₂₀ cycloalkylalkyl, e.g., the alkanyl, alkenyl, or alkynyl moiety of the cycloalkylalkyl group is C₄₋₈ and the cycloalkyl moiety is C_{4-12.} In certain embodiments, a cycloalkylalkyl group is C₄₋₁₂ cycloalkylalkyl.

“Disease” refers to a disease, disorder, condition, or symptom of any of the foregoing.

“Dosage form” refers to a form of a formulation that contains an amount of active agent or prodrug of an active agent, e.g., the R-baclofen prodrug (1), which can be administered to a patient to achieve a therapeutic effect. An oral dosage form is intended to be administered to a patient via the mouth and swallowed. Examples of oral dosage forms include capsules, tablets, and liquid suspensions. A dose of a drug may include one or more dosage forms administered simultaneously or over a period of time.

“Drug” as defined under 21 U.S.C. § 321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . ”

“Heteroalkyl” by itself or as part of another substituent refer to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—NR—, ═N—N═, —═N—, —N═N—NR¹³—, —PR¹³—, —P(O)₂—, —POR¹³—, —O—P(O)₂—, —SO—, —SO₂—, —Sn(R¹³)₂—, and the like, where each R¹³ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R¹³ is independently chosen from hydrogen and C_{1.3} alkyl. In certain embodiments, a heteroatomic group is chosen from —O—, —S—, —NH—, —N(CH₃)—, and —SO₂—; and in certain embodiments, the heteroatomic group is —P—.

“Heteroaryl” refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

“Heterocycloalkyl” refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, a heterocycloalkyl group is C₄₋₁₀ heterocycloalkyl, C₄₋₈ heterocycloalkyl, and in certain embodiments, C₄₋₈ heterocycloalkyl.

“Immediate release” refers to formulations or dosage forms that rapidly dissolve in vitro and in vivo and are intended to be completely dissolved and absorbed in the stomach or upper gastrointestinal tract. Immediate release formulations can release at least 90% of the active ingredient or precursor thereof within about 15 minutes, within about 30 minutes, within about one hour, or within about two hours of administering an immediate release dosage form.

“Leaving group” has the meaning conventionally associated with it in synthetic organic chemistry, i.e., an atom or a group capable of being displaced by a nucleophile and includes halogen such as chloro, bromo, fluoro, and iodo; acyloxy, such as acetoxy and benzoyloxy, alkoxycarbonylaryloxycarbonyl, mesyloxy, tosyloxy, and trifluoromethanesulfonyloxy; aryloxy such as 2,4-dinitrophenoxy, methoxy, N,O-dimethylhydroxylamino, p-nitrophenolate, imidazolyl, and the like.

“MHF” refers to methyl hydrogen fumarate, a compound having the following chemical structure:

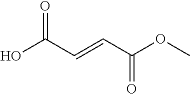

This compound is also sometimes referred to as monomethyl fumarate (MMF).

“MHF Prodrug” refers to a prodrug that is metabolized in vivo to form methyl hydrogen fumarate as a pharmacologically active metabolite.

“Parent heteroaromatic ring system” refers to an aromatic ring system in which one or more carbon atoms (and any associated hydrogen atoms) are independently replaced with the same or different heteroatom in such a way as to maintain the continuous TT-electron system characteristic of aromatic systems and a number of out-of-plane TT-electrons corresponding to the Hückel rule (4n+2). Examples of heteroatoms to replace the carbon atoms include, for example, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, for example, arsindole, carbazole, p-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound that possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N methylglucamine, and the like. In certain embodiments, a pharmaceutically acceptable salt is the hydrochloride salt. In certain embodiments, a pharmaceutically acceptable salt is the sodium salt.

“Pharmaceutically acceptable excipient” refers to a pharmaceutically acceptable filler, a pharmaceutically acceptable adjuvant, a pharmaceutically acceptable vehicle, a pharmaceutically acceptable carrier, or a combination of any of the foregoing with which a compound provided by the present disclosure may be administered to a patient, which does not destroy the pharmacological activity thereof and which is non-toxic when administered in doses sufficient to provide a therapeutically effective amount of the compound or a pharmacologically active metabolite thereof.

“Prodrug” refers to a compound administered in a pharmacologically inactive (or significantly less active) form. Once administered, the compound is metabolized in vivo into an active metabolite. Prodrugs may be designed to improve oral bioavailability, particularly in cases where the metabolite exhibits poor absorption from the gastrointestinal tract. Prodrugs can be used to optimize the absorption, distribution, metabolism, and excretion (ADME) of the active metabolite.

A composition or material that is “substantially free of carboxylic acid moieties” is a composition or material that has less than 2% w/w of carboxylic acid moieties. In certain embodiments, a composition or material that is “substantially free of carboxylic acid moieties” is a composition or material that has less than 1% w/w of carboxylic acid moieties. In certain embodiments, a composition or material that is “substantially free of carboxylic acid moieties” is a composition or material that has less than 0.01% w/w of carboxylic acid moieties.

“Substituent” refers to a group in which one or more hydrogen atoms are independently replaced (or substituted) with the same or substituent group(s). In certain embodiments, each substituent group is independently chosen from halogen, —OH, —CN, —CF₃, ═O, —NO₂, benzyl, —C(O)NH₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, —NO₂, benzyl, —R¹¹, —OR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from —OH, C₁₋₄ alkyl, and —NH₂.

“Sustained-release” refers to release of a drug from a dosage form in which the drug release occurs over a period of time. Sustained release can mean that release of the drug from the dosage form is extended for longer than it would be in an immediate-release dosage form, i.e., at least over several hours. In some embodiments, in vivo release of the compound occurs over a period of at least 2 hours, in some embodiments, over a period of at least about 4 hours, in some embodiments, over a period of at least about 8 hours, in some embodiments over a period of at least about 12 hours, in some embodiments, over a period of at least about 16 hours, in some embodiments, over a period of at least about 20 hours, and in some embodiments, over a period of at least about 24 hours.

“Treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to protecting against or delaying the onset of at least one or more symptoms of a disease in a patient.

“Therapeutically effective amount” refers to the amount of a compound that, when administered to a subject for treating a disease, or at least one of the clinical symptoms of a disease, is sufficient to effect such treatment of the disease or symptom thereof. The “therapeutically effective amount” may vary depending, for example, on the compound, the disease and/or symptoms of the disease, severity of the disease and/or symptoms of the disease, the age, weight, and/or health of the patient to be treated, and the judgment of the prescribing physician. An appropriate amount in any given compound may be ascertained by those skilled in the art and/or is capable of determination by routine experimentation.

“Therapeutically effective dose” refers to a dose that provides effective treatment of a disease in a patient. A therapeutically effective dose may vary from compound to compound and/or from patient to patient, and may depend upon factors such as the condition of the patient and the severity of the disease. A therapeutically effective dose may be determined in accordance with routine pharmacological procedures known to those skilled in the art.

Reference is now made in detail to certain embodiments of compounds, compositions, and methods. The disclosed embodiments are not intended to be limiting of the claims. To the contrary, the claims are intended to cover all alternatives, modifications, and equivalents.

### DETAILED DESCRIPTION

The oral pharmaceutical compositions disclosed herein are so-called tablet-in-tablet compositions. In general, the tablet-in-tablet compositions described herein are produced by first preparing a tablet core from a first component, and then applying during a subsequent compression step a compression coating layer (which is sometimes referred to as a shell or mantle) of a second component in a manner such that the finished formulation comprises the core surrounded by the compression coating. Tablet-in-tablet compositions are disclosed for example in U.S. Pat. Nos. 8, 148,393; 8,088,786; 8,067,033; 7,195,769; and 6,770,297.

### A. Tablet Core

The dosage forms disclosed herein include a tablet core containing a compound selected from (i) methyl hydrogen fumarate (MHF), (ii) a prodrug of methyl hydrogen fumarate, (iii) pharmaceutically acceptable salts of (i) and (ii), and (iv) combinations thereof. Compressed tablet cores containing a fumarate compound can be made using well-known techniques such as those described in Remington: The Science and Practice of Pharmacy, 21^{st} Edition, University of the Sciences in Philadelphia Ed. (2005). Such tablet cores can contain one or more known tableting excipients such as binders, fillers, disintegrants, glidants, lubricants, surfactants, plasticizers, anti-adherents, buffers, disintegrants, wetting agents, emulsifying agents, thickening agents, coloring agents, sustained release agents, or combinations of any of the foregoing. In certain embodiments, the excipient is substantially free of carboxylic acid moieties.

Binders may be included in the tablet core to hold the components of the core together. Examples of binders useful in the present disclosure include, for example, polyvinylpyrrolidone, hydroxypropyl cellulose, hydroxypropyl methyl cellulose, methylcellulose, hydroxyethyl cellulose, sugars, dextran, cornstarch, and combinations of any of the foregoing. In certain embodiments, the binder is hydroxypropyl cellulose. Fillers may be added to increase the bulk to make dosage forms. Examples of fillers useful in the present disclosure include dibasic calcium phosphate, dibasic calcium phosphate dihydrate, calcium sulfate, dicalcium phosphate, tricalcium phosphate, lactose, cellulose including microcrystalline cellulose, mannitol, sodium chloride, dry starch, pregelatinized starch, compressible sugar, mannitol, and combinations of any of the foregoing. In certain embodiments, the filler is lactose monohydrate. Fillers may be water insoluble, water soluble, or combinations thereof. Examples of useful water insoluble fillers include starch, dibasic calcium phosphate dihydrate, calcium sulfate, dicalcium phosphate, tricalcium phosphate, powdered cellulose, microcrystalline cellulose, and combinations of any of the foregoing. Examples of water-soluble fillers include water soluble sugars and sugar alcohols, such as lactose, glucose, fructose, sucrose, mannose, dextrose, galactose, the corresponding sugar alcohols and other sugar alcohols, such as mannitol, sorbitol, xylitol, and combinations of any of the foregoing. In certain embodiments wherein the filler is lactose, a tablet dosage form may comprise an amount of filler ranging from about 25 wt % to about 60 wt %, and in certain embodiments, from about 30 wt % to about 55 wt %.

Glidants may be included in the tablet core to reduce sticking effects during processing, film formation, and/or drying. Examples of useful glidants include talc, magnesium stearate, glycerol monostearate, colloidal silicon dioxide, precipitated silicon dioxide, fumed silicon dioxide, and combinations of any of the foregoing. In certain embodiments, a glidant is colloidal silicon dioxide. Tablet dosage forms may comprise less than about 3 wt % of a glidant, in certain embodiments, less than about 1 wt % of a glidant as a flow aid.

Lubricants and anti-static agents may be included in a pharmaceutically acceptable coating to aid in processing. Examples of lubricants useful in coatings provided by the present disclosure include calcium stearate, glycerol behenate, glyceryl monostearate, magnesium stearate, mineral oil, polyethylene glycol, sodium stearyl fumarate, sodium lauryl sulfate, stearic acid, talc, vegetable oil, zinc stearate, and combinations of any of the foregoing. In certain embodiments, the lubricant is magnesium stearate. In certain embodiments, oral dosage forms may comprise an amount of lubricant ranging from about 0.5 wt % to about 3 wt %.

### 1. Immediate Release Formulations in the Core

In various embodiments, the core contains an immediate release formulation of the active compound. The immediate release formulation can be any immediate release formulation known in the art. Various immediate release formulations include uncoated active compound, immediate release particles, granules or powders of the compound, inert cores having a coating of the compound, and/or granules or pellets of the compound coated with a highly soluble immediate release coating.

In certain embodiments, immediate release particles may comprise the compound and any appropriate vehicle, for example, any of those disclosed herein. The compound is combined with any tableting excipient known in the art to allow release of the compound as an immediate release formulation.

Disintegrants may be included in the tablet core to cause a tablet core to break apart, for example, by expansion of a disintegrants when exposed to water. Examples of useful disintegrants include water swellable substances such as croscarmellose sodium, sodium starch glycolate, cross-linked polyvinyl pyrrolidone, and combinations of any of the foregoing. In various embodiments, the disintegrants can be selected to be substantially free of carboxylic acid moieties.

In various embodiments, immediate release formulations can include granules of the compound formed by granulation methods known to those skilled in the art.

### 2. Sustained Release in the Core

The tablet core may also be formulated in a sustained release formulation. Examples of materials for effecting sustained release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropyl methyl cellulose phthalate, methylcellulose, ethyl cellulose, cellulose acetate, cellulose acetate phthalate, cellulose acetate trimellitate, and carboxymethylcellulose sodium; acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymers, polyacrylamide, aminoalkyl methacrylate copolymer, poly(methacrylic acid anhydride), glycidyl methacrylate copolymers, ammonioalkyl methacrylate copolymers, and methacrylic resins commercially available under the tradename Eudragit® including Eudragit® L, Eudragit® S, Eudragit®E, Eudragit® RL, and Eudragit® RS; vinyl polymers and copolymers such as polyvinylpyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylase, and guar gum; and shellac. Combinations of any of the foregoing polymers may also be used to form sustained-release coatings.

### 3. Compression Coated Core

The core can comprise one or more the components described for the compression coating layer, infra. In such formulations, the core reduces the amount of fumarate compound in the core from being released. As such, the core can have any of the components and properties of the compression coating layer as described herein. It is understood that in such variations, the compression coating core and compression coating layer may have the same or different components.

### B. Compression Coating Layer

A compression coating layer surrounds the core of the tablet dosage form. The function of the coating layer is to reduce the amount of the fumarate compound contained in the core being released from the tablet while the dosage form remains in the patient's stomach. Typically it takes from 1 to 3 hours, measured from the time of swallowing, for the contents of the stomach to pass into the small (upper) intestine. Thus, the coating layer comprises a material that releases no more than 20% of the compound over a period of 2 hours after the tablet is placed in an aqueous solution free of the compound. In other embodiments, the coating layer comprises a material that releases no more than 10% of the compound over a period of 2 hours after the tablet is placed in an aqueous solution free of the compound. In this way, the coating layer reduces the amount of fumarate compound coming into contact with the epithelial tissues lining the stomach.

The protective function of the compression coating layer is achieved by appropriate selection of the coating layer material as well as the thickness. Generally, the coating layer thickness can be expressed as a weight percent of the total tablet, or as a weight ratio of the tablet coating to the tablet core. In certain aspects, the tablets can have a core weight to: compressed coating weight ratio of 1:1 to 1:3. Similarly, in certain aspects the tablets can have a compression coating that ranges from about 40 wt % to about 75 wt % of the total tablet weight.

In various aspects, the oral pharmaceutical tablet is configured to release at least 80% of the compound within 3 hours after being placed in an aqueous solution free of the compound. In further embodiments, the oral pharmaceutical tablet is configured to release at least 80% of the compound over a period of at least 6 hours after being placed in an aqueous solution free of the compound.

In various aspects, the rate of erosion of the compression coating layer can be reduced by increasing the amount of erodible material in the compression coating layer, and/or increasing the viscosity of the erodible material. By increasing the amount of the erodible material in the compression coating layer, the compression coating layer requires more time to erode, and thereby releases less active compound over time.

Likewise, the viscosity of erodible materials in the compression coating layer can be increased. By increasing the viscosity of the erodible material in the compression coating layer, the compression coating layer requires more time to erode and releases less active compound over time. Various erodible materials have a range of viscosities depending on structural properties such as polymer molecular weight, the degree of crosslinking, etc. For example, polymers can be obtained with a range of increasing viscosities. Viscosities of various materials can be obtained from Rowe, Raymond C, Paul J. Sheskey, and Paul J. Weller. Handbook of Pharmaceutical Excipients. London: Pharmaceutical Press, 2003. The oral pharmaceutical tablet can be configured to result in a therapeutic concentration of MHF in blood plasma of the patient of at least 0.7 μg/ml at a time within 24 hours after said oral administration. In further embodiments, the oral pharmaceutical tablet is configured to result in an area under a concentration of methyl hydrogen fumarate in blood plasma versus time curve (AUC) of at least 12.0 μg·hr/ml over 24 hours after start of the oral administration.

In certain embodiments, there is sufficient coating material to cover the outer surface of the tablet core. In certain embodiments, the tablet has a core weight to compressed coating weight ratio of 1:1 to 1:3. The coating layer is sufficiently thick such that the coating layer releases no more than 20% of the compound over a period of 2 hours after the tablet is placed in an aqueous free of the compound. The thickness can depend on the material composition of the coating layer. In certain embodiments, the coating layer thickness is equal to or greater than 0.5 mm. The coating layer is sufficiently thick to cover the entire outer surface of the tablet core.

### Compression Coating Layer Materials

A compression coating layer surrounds the tablet core. It will be understood that either the compression coating layer is in direct contact with the tablet core, or that one or more intermediate layers are disposed between the compression coating layer and the tablet core.

The compression coating comprises one or more materials that will not cause premature breakdown of the fumarate compound during product shelf life.

Surprisingly, MHF and MHF prodrugs have been found to have poor stability when placed in contact with ionizable polymers having carboxylic acid moieties of the type that are commonly used in enteric coatings. Such enteric polymers include for example hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate. Thus, in certain embodiments the compression coating is substantially free of ionizable polymers having carboxylic acid moieties of the types mentioned above.

Compression coating layers release no more than 20% of the compound over a period of 2 hours after the tablet is placed in an aqueous solution free of the compound.

### 1. Non-Ionizable Polymers

As used herein, non-ionizable polymers are materials that are either (i) a proton-donating acidic material having a pKa of greater than 8, or (ii) a proton-accepting basic material having a pKa of less than 2.

In some variations, the compression coating layer comprises a material that is (i) a proton-donating acidic material having a pKa of greater than 8, (ii) a proton-accepting basic material having a pKa of less than 2, (iii) a natural gum or polysaccharide, (iv) a neutral polymer salt, (v) a sugar, or (vi) a lipid. In certain embodiments, the compression coating layer comprises a material that is either (i) a proton-donating acidic material having a pKa of greater than 10, or (ii) a proton-accepting basic material having a pKa of less than 0. The pKa values for various compounds may be calculated as is known in the art.

The compression coating layer can be comprised of one or more non-ionizable polymers. Examples of suitable non-ionizable polymers include non-ionizable cellulosic polymers, non-ionizable vinyl and polyvinyl alcohol polymers, non-ionizable polymers that are not cellulose or vinyl-based, natural gum and polysaccharides, neutral polymer salts, readily ionizable polymers lacking carboxylic acid moieties, and lipids.

a. Non-Ionizable Cellulosic Polymers

In some variations, the compression coating layer comprises a non-ionizable cellulosic polymer. Specific examples of non-ionizable cellulosic polymers include methylcellulose, ethylcellulose, propylcellulose, butylcellulose, cellulose acetate, cellulose propionate, cellulose butyrate, cellulose acetate butyrate, cellulose acetate propionate, methyl cellulose, methyl cellulose acetate, methyl cellulose propionate, methyl cellulose butyrate, ethyl cellulose acetate, ethyl cellulose propionate, ethyl cellulose butyrate, hydroxymethyl cellulose, hydroxyethyl cellulose, hydroxyethyl methyl cellulose, hydroxypropyl cellulose, hydroxybutyl cellulose, hydroxyethyl cellulose acetate, and hydroxyethyl ethyl cellulose, low-substituted hydroxypropyl cellulose, hydroxypropyl methyl cellulose, hydroxypropyl methyl cellulose acetate, hydroxypropyl methyl cellulose propionate, hydroxypropyl methyl cellulose butyrate, and corresponding salts and esters. In general, such non-ionizable cellulosic polymers are substantially free of carboxylic acid moieties.

b. Non-Ionizable Vinyl-Based Polymers

In some variations, the compression coating layer comprises a non-ionizable vinyl-based polymer. Exemplary vinyl-based polymers include polyvinvyl acetate, and polyvinylpyrrolidone. Exemplary vinyl-containing polymers further include vinyl polymers and copolymers having at least hydroxyl-containing repeat units, alkylacyloxy-containing repeat units, or cyclicamido-containing repeat units. Still further exemplary vinyl-containing polymers also include polyvinyl alcohols that have at least a portion of their repeat units in the unhydrolyzed (vinyl acetate) form, polyvinylhydroxyethyl ether, polyvinyl alcohol polyvinyl acetate copolymers, polyvinyl pyrrolidone, polyvinylpyrrolidone-polyvinvylacetate copolymers, polyethylene polyvinyl alcohol copolymers, and polyoxyethylene-polyoxypropylene copolymers. In alternate embodiments, vinyl copolymers can include a second polymer having (1) substantially carboxy-free hydroxyl-containing repeat units and (2) hydrophobic repeat units. In various embodiments, the preceding vinyl-based non-ionizable polymers and co-polymers are substantially free of carboxylic acid moieties.

In certain embodiments, the non-ionizable polyvinyl materials show no degradation as an excipient. Non-limiting examples of such materials include polyvinylpyrrolidone and crospovidone.

c. Non-Ionizable Polymer that is neither Cellulose nor Vinyl Based

In some variations, the compression coating layer comprises non-cellulosic non-vinyl-based non-ionizable polymers. Examples of such polymers include poly(lactide) poly(glycolide), poly(ε-caprolactone), poly(lactide-co-glycolide), poly(lactide-co-ε-caprolactone), poly(ethylene oxide-co-ε-caprolactone), poly(ethylene oxide-co-lactide), poly(ethylene oxide-co-lactide-co-glycolide), poly(isobutyl)cyanoacrylate, and poly(hexyl)cyanoacrylate, polyethylene oxide, and poly(ethyl acrylate-co-methyl methacrylate) 2:1 (Eudragit NE). In some variations, non-ionizable polymers such as polyoxyethylene-polyoxypropylene block copolymers show no degradation as an excipient. In certain variations, the non-cellulosic non-vinyl based non-ionizable polymers are substantially free of carboxylic acid moieties.

In further variations, non-vinyl non-cellulosic non-ionizable polymers and co-polymers are functionalized with one or more carboxyl or amine substituents. Such polymers and co-polymers include carboxylic acid functionalized polymethyacrylates, carboxylic acid functionalized polyacrylate, amine-functionalized polyacrylates, amine-functionalized polymethacrylates, proteins, and carboxylic acid functionalized starches.

### 2. Natural Gums and Polysaccharides

In some variations, the compression coating layer comprises a natural gum or polysaccharides. Suitable examples of such natural gums and polysaccharides include guar gum, tara gum, locust bean gum, carrageenan, gellan gum, alginate, and xanthan gum. In certain embodiments, the natural gums and polysaccharides are substantially free of carboxylic acid moieties, including salts thereof. Non-limiting examples of such materials include guar gum, tara gum, locust bean gum, and carrageenan. In certain embodiments, the natural gums and polysaccharides have carboxylic acid moieties. Non-limiting examples of such materials include gellan gum, Alginate, and xanthan gum.

### 3. Neutral Polymer Salts

In some variations, the compression coating layer comprises a neutral polymer salt. Non-limiting examples of such neutral polymer salts include poly(ethyl acrylate-co-methyl methacrylate-co-trimethylammonioethyl methacrylate chloride) 1:2:0.1, poly(ethyl acrylate-co-methyl methacrylate-co-trimethylammonioethyl methacrylate chloride) 1:2:0.2, crosslinked sodium carboxymethyl cellulose (croscarmellose sodium), crosslinked sodium carboxymethyl cellulose (sodium starch glycolate), salts of carboxymethyl cellulose, salts of carboxyethyl cellulose, salts of carboxypropyl cellulose, salts of carboxybutyl cellulose, salts of carboxymethyl starch, and salts of carboxyethyl starch.

In certain embodiments, the neutral polymer salts are substantially free of carboxylate moieties. Non-limiting examples of such neutral polymer salts include poly(ethyl acrylate-co-methyl methacrylate-co-trimethylammonioethyl methacrylate chloride) 1:2:0.1 and poly(ethyl acrylate-co-methyl methacrylate-co-trimethylammonioethyl methacrylate chloride) 1:2:0.2, In certain additional embodiments, the neutral polymer salts are salts of carboxylate moieties. Non-limiting examples of such neutral polymer salts include crosslinked sodium carboxymethyl cellulose (croscarmellose sodium), crosslinked sodium carboxymethyl cellulose (sodium starch glycolate), salts of carboxymethyl cellulose, salts of carboxyethyl cellulose, salts of carboxypropyl cellulose, salts of carboxybutyl cellulose, salts of carboxymethyl starch, and salts of carboxyethyl starch.

In certain embodiments, the neutral polymer salts do not degrade as excipients. Non-limiting examples of such materials include poly(ethyl acrylate-co-methyl methacrylate-co-trimethylammonioethyl methacrylate chloride) 1:2:0.1 and poly(ethyl acrylate-co-methyl methacrylate-co-trimethylammonioethyl methacrylate chloride) 1:2:0.2, and croscarmellose sodium.

In certain embodiments, certain neutral polymer salts include a carboxyl group that is neutralized with a counter ion. For example, croscarmellose sodium includes a carboxyl group that is neutralized with sodium.

In certain other embodiments, the readily ionizable polymers do not contain carboxylic acid groups. Such materials include poly(butyl methacrylate-co-(2-dimethylaminoethyl) methacrylate-co-methyl methacrylate) 1:2:1 (Eudragit E), chitosan, and methyl methacrylate diethylaminoethyl methacrylate copolymer. Eudragit E has polymer free amino groups, and is neutral at pH>5 and protonated at pH<5. It is therefore soluble in an aqueous solution at low pH and insoluble in an aqueous solution at high pH.

In certain variations, the neutral polymer salts are substantially free of carboxylic acid moieties.

### 4. Lipids

In some variations, the compression coating layer comprises a lipid. Examples of suitable lipids are glyceryl behenate, castor oil, hydrogenated vegetable oil, hydrogenated carnauba wax. and microcrystalline wax. In certain variations, the lipids are substantially free of carboxylic acid moieties.

### Fumarate Compounds; MHF and MHF Prodrugs

In certain embodiments, the active ingredient in the dosage forms disclosed herein is methyl hydrogen fumarate or a pharmaceutically acceptable salt thereof.

Alternatively, the active ingredient in the dosage forms disclosed herein can be an MHF prodrug. One suitable MHF prodrug is dimethyl fumarate. Other suitable MHF prodrugs are the compounds of Formula (I):

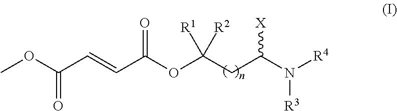

or a pharmaceutically acceptable salt thereof, wherein:
R¹ and R² are independently chosen from hydrogen, C₁₋₆ alkyl, and substituted C₁₋₆ alkyl;
R³ and R⁴ are independently chosen from hydrogen, C₁₋₆ alkyl, substituted C₁₋₆ alkyl, C₁₋₆ heteroalkyl, substituted C₁₋₆ heteroalkyl, C₃₋₁₁ cycloalkyl, substituted C₃₋₁₁ cycloalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R3 and R⁴ together with the nitrogen to which they are bonded form a ring chosen from a C₄₋₁₀ heteroaryl, substituted C₄₋₁₀ heteroaryl, C₄₋₁₀ heterocycloalkyl, and substituted C₄₋₁₀ heterocycloalkyl;
n is an integer from 0 to 4; and
X is independently chosen from a single oxygen atom and a pair of hydrogen atoms; wherein each substituent group is independently chosen from halogen, —OH, —CN, —CF₃, ═O, —O₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl;
and wherein when X is a single oxygen atom, the oxygen atom is connected to the carbon to which it is bonded by a double bond to form a carboxyl group and when X is a pair of hydrogen atoms, each hydrogen atom is connected to the carbon to which it is bonded to by single bond.

Compounds of Formula I are disclosed in (i) Gangakhedkar et al., U.S. Pat. No. 8, 148,414; and (ii) Virsik et al. U.S. Ser. No. 61/653,375, filed May 30, 2012, the disclosures of which are incorporated herein by reference. The methods and schemes of synthesis disclosed in Gangakhedkar et al., U.S. Pat. No. 8,148,414 are incorporated herein by reference. In other embodiments, the MHF prodrug is dimethyl fumarate.

In other embodiments, the MHF prodrug is a compound of Formula (II):

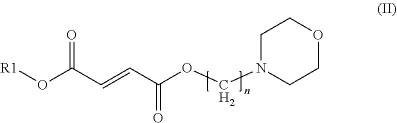

or a pharmaceutically acceptable salt thereof, wherein:
n is an integer from 2 to 6; and
R¹ is methyl.

Compounds of Formula (II) are disclosed in Cundy et al., U.S. Patent Application No. 61/595,835 filed Feb. 7, 2012, the disclosures of which are incorporated herein by reference.

### Therapeutic Uses

The dosage forms disclosed herein may be administered to a patient suffering from any disease including a disorder, condition, or symptom for which MHF is known or hereafter discovered to be therapeutically effective. Indications for which MHF has been prescribed, and hence for which a dosage form disclosed herein is also expected to be effective, include psoriasis. Other indications for which the disclosed dosage forms may be therapeutically effective include multiple sclerosis, an inflammatory bowel disease, asthma, chronic obstructive pulmonary disease, and arthritis.

Methods of treating a disease in a patient provided by the present disclosure comprise administering to a patient in need of such treatment a dosage form disclosed herein. The dosage forms disclosed herein may provide therapeutic or prophylactic plasma and/or blood concentrations of MHF following administration to a patient.

The dosage forms disclosed herein may be administered in an amount and using a dosing schedule as appropriate for treatment of a particular disease. For example, daily doses of

MHF or a MHF prodrug may range from about 0.01 mg/kg to about 50 mg/kg, from about 0.1 mg/kg to about 50 mg/kg, from about 1 mg/kg to about 50 mg/kg, and in certain embodiments, from about 5 mg/kg to about 25 mg/kg. In certain embodiments, the MHF or MHF prodrug may be administered at a dose over time from about 1 mg to about 5 g per day, from about 10 mg to about 4 g per day, and in certain embodiments from about 20 mg to about 2 g per day. An appropriate dose of MHF or a MHF prodrug may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

MHF or a MHF prodrug may be assayed in vitro and in vivo for the desired therapeutic or prophylactic activity prior to use in humans. In vivo assays, for example using appropriate animal models, may also be used to determine whether administration of MHF or a MHF prodrug is therapeutically effective.

In certain embodiments, a therapeutically effective dose of MHF or a MHF prodrug may provide therapeutic benefit without causing substantial toxicity including adverse side effects. Toxicity of MHF or a MHF prodrug and/or metabolites thereof may be determined using standard pharmaceutical procedures and may be ascertained by those skilled in the art. The dose ratio between toxic and therapeutic effect is the therapeutic index. A dose of MHF or a MHF prodrug may be within a range capable of establishing and maintaining a therapeutically effective circulating plasma and/or blood concentration of MHF or a MHF prodrug that exhibits little or no toxicity.

The dosage forms disclosed herein may be used to treat diseases, disorders, conditions, and symptoms of any of the foregoing for which MHF is known to provide or is later found to provide therapeutic benefit. MHF is known to be effective in treating psoriasis, multiple sclerosis, an inflammatory bowel disease, asthma, chronic obstructive pulmonary disease, and arthritis. Hence, the dosage forms disclosed herein may be used to treat any of the foregoing diseases and disorders. The underlying etiology of any of the foregoing diseases being treated may have a multiplicity of origins. Further, in certain embodiments, a therapeutically effective amount of MHF and/or a MHF prodrug may be administered to a patient, such as a human, as a preventative measure against various diseases or disorders. Thus, a therapeutically effective amount of MHF or a MHF prodrug may be administered as a preventative measure to a patient having a predisposition for and/or history of immunological, autoimmune, and/or inflammatory diseases including psoriasis, asthma and chronic obstructive pulmonary diseases, cardiac insufficiency including left ventricular insufficiency, myocardial infarction and angina pectoris, mitochondrial and neurodegenerative diseases such as Parkinson's disease, Alzheimer's disease, Huntington's disease, retinopathia pigmentosa and mitochondrial encephalomyopathy, transplantation rejection, autoimmune diseases including multiple sclerosis, ischemia and reperfusion injury, AGE-induced genome damage, inflammatory bowel diseases such as Crohn's disease and ulcerative colitis; and NF-κB mediated diseases.

### Psoriasis

Psoriasis is a chronic T-cell-mediated autoimmune disease that affects 2% to 3% of the US population and 0.6% to 6.5% of the European population. As many as an estimated 7.5 million Americans are thought to have psoriasis, making it the most common autoimmune disease in the United States (National Psoriasis Foundation). The most common phenotypic form of psoriasis, plaque psoriasis, manifests as raised, well-demarcated, red plaques with silver scales that may remit spontaneously or recur in response to environmental triggers.

In addition to the appearance of skin plaques, there is a high degree of physical morbidity associated with psoriasis. Up to 30% of patients develop psoriatic arthritis. Individuals with psoriasis appear to be at higher risk for a number of co-morbid conditions including obesity, myocardial infarction, cancer, diabetes mellitus, Crohn's disease and depression. Patients with severe psoriasis, on average, die five years younger than patients without psoriasis, an observation that is largely attributable to an increased risk of cardiovascular disease. In addition, psoriasis can represent both a significant social and financial burden for patients. Individuals suffer from both disfigurement and social stigmatization, with an impact on mental disability comparable with that of other major medical illnesses such as heart disease and rheumatoid arthritis. Patients with psoriasis, like those with other major medical disorders, have a reduced quality of life (QoL) and reduced levels of employment and income.

Current treatment is aimed at reducing the burden of disease and achieving an improvement in its signs and symptoms. Topical agents are generally prescribed for mild and some moderate cases, while systemic agents (including biologics), and phototherapy are reserved for moderate and severe cases. Treatment decisions are based not only on the severity of the disease, but also on existing comorbidities, tolerability, side effects, and patient preferences. Several systemic agents, including methotrexate (MTX), cyclosporine, and retinoids are most commonly used. Various factors limit favorable long-term outcomes; in particular, a lack of consistent efficacy over time and the risk of serious cumulative toxicity. Injectable biologic agents are effective for a substantial number of patients, but there is concern regarding long-term safety due to the risk of immunosuppression and the potential for development of infections, opportunistic diseases, or malignancy.

Psoriasis is characterized by hyperkeratosis and thickening of the epidermis as well as by increased vascularity and infiltration of inflammatory cells in the dermis. Psoriasis vulgaris manifests as silvery, scaly, erythematous plaques on typically the scalp, elbows, knees, and buttocks. Guttate psoriasis occurs as tear-drop size lesions.

Fumaric acid esters are recognized for the treatment of psoriasis and dimethyl fumarate is approved for the systemic treatment of psoriasis in Germany (Mrowietz and Asadullah, Trends Mol Med 2005, 11(1), 43-48; and Mrowietz et al., Br J Dermatology 1999, 141, 424-429).

Efficacy of MHF or a MHF prodrug for treating psoriasis can be determined using animal models and in clinical trials.

In a particular embodiment, a method for treating psoriasis in a patient comprises administering a dosage form described herein to said patient.

The period of treatment can vary. In one embodiment, treatment is carried out for at least three months, such as, for example, four months, five months, six months, seven months, eight months, nine months, ten months, eleven months or twelve months.

In a particular embodiment, the psoriasis is stable, moderate-to-severe plaque psoriasis.

The severity of psoriasis can be measured by the Psoriasis Area and Severity Index “PASI”. The PASI is a measure of the average redness, thickness, and scaliness of the lesions (each graded on a 0-4 scale) and weighted by the area of involvement. National Psoriasis Foundation Medical Advisory Board standardized the definitions of events of psoriasis recurrence based on PASI, including the definition of relapse, which is a loss of at least 50% of PASI improvement after discontinuation of treatment in patients who had achieved a clinically meaningful response; and rebound, which is PASI ≥125% of Baseline or new generalized pustular, erythrodermic, or more inflammatory psoriasis (Carey, 2006; Gordon, 2005).

In the current trial, PASI improvement will be evaluated from Baseline to all visits during the Titration and Treatment Periods, and clinically meaningful response is defined as PASI-50 (Carlin, 2004).

In one embodiment, administration of a dosage form described herein provides at least about a 25% reduction in the baseline PASI score (PASI-25), at least about a 50% reduction in the baseline PASI score (PASI-50), at least about a 75% reduction in the baseline PASI score (PASI-75) or at least about a 85% reduction in the baseline PASI score (PASI-80) over the period of treatment.

Another measure of psoriasis severity is the Investigator's Global Assessment (IGA):

| Score | Grade | Definintion |
|---|---|---|
| 0 | Clear | No signs of psoriasis |
| | | Post-inflammatory hyperpigmentation may be |
| | | present |
| 1 | Almost | No thickening to minimal plaque elevation |
| | clear | Normal to slight pink coloration/faint erythema |
| | | Focal to minimal scaling |
| 2 | Mild | Slight elevation/thickening |
| | | Pink to light red coloration |
| | | Predominantly fine scaling partially or mostly |
| | | covering lesions |
| 3 | Moderate | Clearly distinguishable/distinct thickening |
| | | Definite red coloration |
| | | Coarse scaling covering most plaques |
| 4 | Severe | Marked thickening with hard/sharp edges |
| | | Bright to deep dark red coloration |
| | | Thick/coarse scaling covering almost all or all |
| | | lesions |

Adapted from: Langley R G B, Feldman S R, Nyirady J, van de Kerkhof P, et al. The 5-point Investigator’s Global Assessment (IGA) Scale: A modified tool for evaluating plaque psoriasis severity in clinical trials. J Dermatolog Treat. 2015; 26(1): 23-31.

In one embodiment, administration of a dosage form described herein provides a reduction in the IGA score over the period of treatment. In a particular embodiment, treatment with a dosage form described herein results in an IGA score of 0 (clear) or 1 (almost clear).

In a particular embodiment, a method for treating psoriasis in a patient comprises administering a sustained release compression coated dosage form comprising the active (N,N-Diethylcarbamoyl)methyl methyl (2E)but-2-ene-1,4-dioate to said patient.

In a particular embodiment, each dosage form contains at least 100 mg (N,N-Diethylcarbamoyl)methyl methyl (2E)but-2-ene-1,4-dioate, at least 200 mg (N,N-Diethylcarbamoyl)methyl methyl (2E)but-2-ene-1,4-dioate at least 300 mg (N,N-Diethylcarbamoyl)methyl methyl (2E)but-2-ene-1,4-dioate, at least 400 mg (N,N-Diethylcarbamoyl)methyl methyl (2E)but-2-ene-1,4-dioate, at least 500 mg (N,N-Diethylcarbamoyl)methyl methyl (2E)but-2-ene-1,4-dioate or at least 600 mg (N,N-Diethylcarbamoyl)methyl methyl (2E)but-2-ene-1,4-dioate. In a more particular embodiment, each dosage form contains about 100 mg (N,N-Diethylcarbamoyl)methyl methyl (2E)but-2-ene-1,4-dioate or about 200 mg (N,N-Diethylcarbamoyl)methyl methyl (2E)but-2-ene-1,4-dioate

In another embodiment, the dosage form is administered once daily, twice daily, three times daily or four times daily.

The total daily dose of (N,N-Diethylcarbamoyl)methyl methyl (2E)but-2-ene-1,4-dioate can vary depending on the amount of active in the dosage form and the frequency of administration. In one embodiment, at least about 400 mg (N,N-Diethylcarbamoyl)methyl methyl (2E)but-2-ene-1,4-dioate is administered daily, such as, for example, at least about 500 mg (N,N-Diethylcarbamoyl)methyl methyl (2E)but-2-ene-1,4-dioate or at least about 600 mg (N,N-Diethylcarbamoyl)methyl methyl (2E)but-2-ene-1,4-dioate. In more particular embodiments, the total daily dosage is about 400 mg (N,N-Diethylcarbamoyl)methyl methyl (2E)but-2-ene-1,4-dioate or about 600 mg (N,N-Diethylcarbamoyl)methyl methyl (2E)but-2-ene-1,4-dioate.

In even more particular embodiments, about 400 mg (N,N-Diethylcarbamoyl)methyl methyl (2E)but-2-ene-1,4-dioate is administered once daily or about 400 mg (N,N-Diethylcarbamoyl)methyl methyl (2E)but-2-ene-1,4-dioate is administered twice daily or about 600 mg (N,N-Diethylcarbamoyl)methyl methyl (2E)but-2-ene-1,4-dioate is administered once daily.

### Inflammatory Arthritis

Inflammatory arthritis includes diseases such as rheumatoid arthritis, juvenile rheumatoid arthritis (juvenile idiopathic arthritis), psoriatic arthritis, and ankylosing spondylitis produce joint inflammation. The pathogenesis of immune-mediated inflammatory diseases including inflammatory arthritis is believed to involve TNF and NK-κB signaling pathways (Tracey et al., Pharmacology & Therapeutics 2008, 117, 244-279). DMF has been shown to inhibit TNF and inflammatory diseases including inflammatory arthritis are believed to involve TNF and NK-κB signaling and therefore may be useful in treating inflammatory arthritis (Lowewe et al., J Immunology 2002, 168, 4781-4787).

The efficacy of MHF or a MHF prodrug for treating inflammatory arthritis can be determined using animal models and in clinical trials.

### Multiple Sclerosis

Multiple sclerosis (MS) is an inflammatory autoimmune disease of the central nervous system caused by an autoimmune attack against the isolating axonal myelin sheets of the central nervous system. Demyelination leads to the breakdown of conduction and to severe disease with destruction of local axons and irreversible neuronal cell death. The symptoms of MS are highly varied with each individual patient exhibiting a particular pattern of motor, sensible, and sensory disturbances. MS is typified pathologically by multiple inflammatory foci, plaques of demyelination, gliosis, and axonal pathology within the brain and spinal cord, all of which contribute to the clinical manifestations of neurological disability (see e.g., Wingerchuk, Lab Invest 2001, 81, 263-281; and Virley, NeuroRx 2005, 2(4), 638-649). Although the causal events that precipitate MS are not fully understood, evidence implicates an autoimmune etiology together with environmental factors, as well as specific genetic predispositions. Functional impairment, disability, and handicap are expressed as paralysis, sensory and octintive disturbances spasticity, tremor, a lack of coordination, and visual impairment, which impact on the quality of life of the individual. The clinical course of MS can vary from individual to individual, but invariably the disease can be categorized in three forms: relapsing-remitting, secondary progressive, and primary progressive.

Studies support the efficacy of FAEs for treating MS and are undergoing phase II clinical testing (Schimrigk et al., Eur J Neurology 2006, 13, 604-610; and Wakkee and Thio, Current Opinion Investigational Drugs 2007, 8(11), 955-962).

Assessment of MS treatment efficacy in clinical trials can be accomplished using tools such as the Expanded Disability Status Scale and the MS Functional as well as magnetic resonance imaging lesion load, biomarkers, and self-reported quality of life. Animal models of

MS shown to be useful to identify and validate potential therapeutics include experimental autoimmune/allergic encephalomyelitis (EAE) rodent models that simulate the clinical and pathological manifestations of MS and nonhuman primate EAE models.

### Inflammatory Bowel Disease (Crohn's Disease, Ulcerative Colitis)

Inflammatory bowel disease (IBD) is a group of inflammatory conditions of the large intestine and in some cases, the small intestine that includes Crohn's disease and ulcerative colitis. Crohn's disease, which is characterized by areas of inflammation with areas of normal lining in between, can affect any part of the gastrointestinal tract from the mouth to the anus. The main gastrointestinal symptoms are abdominal pain, diarrhea, constipation, vomiting, weight loss, and/or weight gain. Crohn's disease can also cause skin rashes, arthritis, and inflammation of the eye. Ulcerative colitis is characterized by ulcers or open sores in the large intestine or colon. The main symptom of ulcerative colitis is typically constant diarrhea with mixed blood of gradual onset. Other types of intestinal bowel disease include collagenous colitis, lymphocytic colitis, ischaemic colitis, diversion colitis, Behcet's colitis, and indeterminate colitis.

FAEs are inhibitors of NF-κB activation and therefore may be useful in treating inflammatory diseases such as Crohn's disease and ulcerative colitis (Atreya et al., J Intern Med 2008, 263(6), 59106).

The efficacy of MHF or a MHF prodrug for treating inflammatory bowel disease can be evaluated using animal models and in clinical trials. Useful animal models of inflammatory bowel disease are known.

### Asthma

Asthma is reversible airway obstruction in which the airway occasionally constricts, becomes inflamed, and is lined with an excessive amount of mucus. Symptoms of asthma include dyspnea, wheezing, chest tightness, and cough. Asthma episodes may be induced by airborne allergens, food allergies, medications, inhaled irritants, physical exercise, respiratory infection, psychological stress, hormonal changes, cold weather, or other factors.

As an inhibitor of NF-κB activation and as shown in animal studies (Joshi et al., US 2007/0027076) FAEs may be useful in treating pulmonary diseases such as asthma and chronic obstructive pulmonary disorder.

The efficacy of MHF or a MHF prodrug for treating asthma can be assessed using animal models and in clinical trials.

### Chronic Obstructive Pulmonary Disease

Chronic obstructive pulmonary disease (COPD), also known as chronic obstructive airway disease, is a group of diseases characterized by the pathological limitation of airflow in the airway that is not fully reversible, and includes conditions such as chronic bronchitis, emphysema, as well as other lung disorders such as asbestosis, pneumoconiosis, and pulmonary neoplasms (see, e.g., Barnes, Pharmacological Reviews 2004, 56(4), 515-548). The airflow limitation is usually progressive and associated with an abnormal inflammatory response of the lungs to noxious particles and gases. COPD is characterized by a shortness of breath the last for months or years, possibly accompanied by wheezing, and a persistent cough with sputum production. COPD is most often caused by tobacco smoking, although it can also be caused by other airborne irritants such as coal dust, asbestos, urban pollution, or solvents. COPD encompasses chronic obstructive bronchiolitis with fibrosis and obstruction of small airways, and emphysema with enlargement of airspaces and destruction of lung parenchyma, loss of lung elasticity, and closure of small airways.

The efficacy of administering MHF or a MHF prodrug for treating chronic obstructive pulmonary disease may be assessed using animal models of chronic obstructive pulmonary disease and in clinical studies. For example, murine models of chronic obstructive pulmonary disease are known.

### Neurodegenerative Disorders

Neurodegenerative diseases such as Parkinson's disease, Alzheimer's disease, Huntington's disease and amyoptrophic lateral sclerosis are characterized by progressive dysfunction and neuronal death. NF-κB inhibition has been proposed as a therapeutic target for neurodegenerative diseases (Camandola and Mattson, Expert Opin Ther Targets 2007, 11(2), 123-32).

### Parkinson's Disease

Parkinson's disease is a slowly progressive degenerative disorder of the nervous system characterized by tremor when muscles are at rest (resting tremor), slowness of voluntary movements, and increased muscle tone (rigidity). In Parkinson's disease, nerve cells in the basal ganglia, e.g., substantia nigra, degenerate, and thereby reduce the production of dopamine and the number of connections between nerve cells in the basal ganglia. As a result, the basal ganglia are unable to smooth muscle movements and coordinate changes in posture as normal, leading to tremor, incoordination, and slowed, reduced movement (bradykinesia) (Blandini, et al., Mol. Neurobiol. 1996, 12, 73-94).

The efficacy of MHF or a MHF prodrug for treating Parkinson's disease may be assessed using animal and human models of Parkinson's disease and in clinical studies.

### Alzheimer's Disease

Alzheimer's disease is a progressive loss of mental function characterized by degeneration of brain tissue, including loss of nerve cells and the development of senile plaques and neurofibrillary tangles. In Alzheimer's disease, parts of the brain degenerate, destroying nerve cells and reducing the responsiveness of the maintaining neurons to neurotransmitters.

Abnormalities in brain tissue consist of senile or neuritic plaques, e.g., clumps of dead nerve cells containing an abnormal, insoluble protein called amyloid, and neurofibrillary tangles, twisted strands of insoluble proteins in the nerve cell.

The efficacy of MHF or a MHF prodrug for treating Alzheimer's disease may be assessed using animal and human models of Alzheimer's disease and in clinical studies.

### Huntington's Disease

Huntington's disease is an autosomal dominant neurodegenerative disorder in which specific cell death occurs in the neostriatum and cortex (Martin, N Engl J Med 1999, 340, 1970-80). Onset usually occurs during the fourth or fifth decade of life, with a mean survival at age of onset of 14 to 20 years. Huntington's disease is universally fatal, and there is no effective treatment. Symptoms include a characteristic movement disorder (Huntington's chorea), cognitive dysfunction, and psychiatric symptoms. The disease is caused by a mutation encoding an abnormal expansion of CAG-encoded polyglutamine repeats in the protein, huntingtin.

The efficacy of MHF or a MHF prodrug for treating Huntington's disease may be assessed using animal and human models of Huntington's disease and in clinical studies.

### Amyotrophic Lateral Sclerosis

Amyotrophic lateral sclerosis (ALS) is a progressive neurodegenerative disorder characterized by the progressive and specific loss of motor neurons in the brain, brain stem, and spinal cord (Rowland and Schneider, N Engl J Med 2001, 344, 1688-1700). ALS begins with weakness, often in the hands and less frequently in the feet that generally progresses up an arm or leg. Over time, weakness increases and spasticity develops characterized by muscle twitching and tightening, followed by muscle spasms and possibly tremors. The average age of onset is 55 years, and the average life expectancy after the clinical onset is 4 years. The only recognized treatment for ALS is riluzole, which can extend survival by only about three months.

The efficacy MHF or a MHF prodrug for treating ALS may be assessed using animal and human models of ALS and in clinical studies.

### Other Diseases

Other diseases and conditions for which MHF or a MHF prodrug such as DMF or a compound of Formulae (I) or (II) can be useful in treating include rheumatica, granuloma annulare, lupus, autoimmune carditis, eczema, sarcoidosis, and autoimmune diseases including acute disseminated encephalomyelitis, Addison's disease, alopecia areata, ankylosing spondylitis, antiphospholipid antibody syndrome, autoimmune hemolytic anemia, autoimmune hepatitis, autoimmune inner ear disease, bullous pemphigoid, Behcet's disease, celiac disease, Chagas disease, chronic obstructive pulmonary disease, Crohn's disease, dermatomyositis, diabetes mellitus type I, endometriosis, Goodpasture's syndrome, Graves' disease, Guillain-Barre syndrome, Hashimoto's disease, hidradenitis suppurativea, Kawasaki disease, IgA neuropathy, idiopathic thrombocytopenia purpura, interstitial cystitis, lupus erythematosus, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, schizophrenia, scleroderma, Sjogren's syndrome, stiff person syndrome, temporal arteritis, ulcerative colitis, vasculitis, vitiligo, Wegener's granulomatosis, optic neuritis, neuromyelitis optica, subacute necrotizing myelopathy, balo concentric sclerosis, transverse myelitis, susac syndrome, central nervous system vasculitis, neurosarcoidosis, Charcott-Marie-Tooth Disease, progressive supranuclear palsy, neurodegeneration with brain iron accumulation, pareneoplastic syndromes, primary lateral sclerosis, Alper's Disease, monomelic myotrophy, adrenal leukodystrophy, Alexanders Disease, Canavan disease, childhood ataxia with central nervous system hypomyelination, Krabbe Disease, Pelizaeus-Merzbacher disease, Schilders Disease, Zellweger's syndrome, Sjorgren's Syndrome, human immunodeficiency viral infection, hepatitis C viral infection, herpes simplex viral infection and a tumor.

### Dosing

The dosage forms disclosed herein, and their use for therapeutic treatment, are not limited to any particular oral dosing regimen as long as the dosing regimen achieves therapeutic blood plasma concentration levels and AUC levels. MHF or a MHF prodrug may be administered at dosage levels of about 0.001 mg/kg to about 50 mg/kg, from about 0.01 mg/kg to about 25 mg/kg, or from about 0.1 mg/kg to about 10 mg/kg of subject body weight per day, one, two, three, four or more times a day, to obtain the desired concentrations and AUC for MHF in the blood plasma.

In various embodiments, the tablet can contain more than 50 mg of prodrug In further embodiments, the tablet can contain more than 100 mg of prodrug. In further embodiments, the tablet can contain more than 150 mg of prodrug. In further embodiments, the tablet can contain more than 200 mg of prodrug. In further embodiments, the tablet can contain more than 250 mg of prodrug. In further embodiments, the tablet can contain more than 300 mg of prodrug. In further embodiments, the tablet can contain more than 350 mg of prodrug. In various embodiments, the oral pharmaceutical tablet can contain equal to or less than 900 mg of prodrug. In further embodiments, the tablet can contain less than 800 mg of prodrug.

In further embodiments, the tablet can contain less than 700 mg of prodrug. In further embodiments, the tablet can contain less than 600 mg of prodrug. In further embodiments, the tablet can contain less than 500 mg of prodrug. In further embodiments, the tablet can contain less than 450 mg of prodrug. In further embodiments, the tablet can contain less than 400 mg of prodrug. In further embodiments, the tablet can contain less than 350 mg of prodrug. In further embodiments, the tablet can contain less than 300 mg of prodrug. In further embodiments, the tablet can contain less than 250 mg of prodrug.

For the treatment of multiple sclerosis and/or psoriasis, blood plasma concentrations of MHF of at least 0.5 μg/ml during the course of dosing is desired. In other embodiments, blood plasma concentrations of MHF of at least 0.7 μg/ml during the course of dosing is desired. In other embodiments, blood plasma concentrations of MHF of at least 1.2 μg/ml during the course of dosing is desired.

Similarly, for the treatment of multiple sclerosis and/or psoriasis, an area under a concentration of MHF in blood plasma versus time curve (AUC) of at least 4.0 μg·hr/ml over 24 hours of dosing is desired. In other embodiments, an area under a concentration of MHF in blood plasma versus time curve (AUC) of at least 4.8 μg·hr/ml over 24 hours of dosing is desired. In other embodiments, an area under a concentration of MHF in blood plasma versus time curve (AUC) of at least 6.0 μg hr/ml over 24 hours of dosing is desired. In other embodiments, an area under a concentration of MHF in blood plasma versus time curve (AUC) of at least 7.0 μg·hr/ml over 24 hours of dosing is desired. In other embodiments, an area under a concentration of MHF in blood plasma versus time curve (AUC) of at least 9.0 μg·hr/ml over 24 hours of dosing is desired. In other embodiments, an area under a concentration of MHF in blood plasma versus time curve (AUC) of at least 10.5 μg·hr/ml over 24 hours of dosing is desired. In still other embodiments, an area under a concentration of MHF in blood plasma versus time curve (AUC) of at least 12.0 μg·hr/ml over 24 hours of dosing is desired.

The amount of MHF or a MHF prodrug that will be effective in the treatment of a disease in a patient will depend, in part, on the nature of the condition and can be determined by standard clinical techniques known in the art. In addition, in vitro or in vivo assays may be employed to help identify optimal dosage ranges. A therapeutically effective amount of MHF or a MHF prodrug to be administered may also depend on, among other factors, the subject being treated, the weight of the subject, the severity of the disease, and the judgment of the prescribing physician.

For oral systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

A dose may be administered in a single dosage form or in multiple dosage forms. When multiple dosage forms are used the amount of compound contained within each dosage form may be the same or different. The amount of MHF or a MHF prodrug contained in a dose may depend on whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. In certain embodiments, a MHF prodrug may exhibit a high therapeutic index. The data obtained from these cell culture assays and animal studies may be used in formulating a dosage range that is not toxic for use in humans. A dose of MHF or a MHF prodrug provided by the present disclosure may be within a range of circulating concentrations in for example the blood, plasma, or central nervous system, that include the effective dose and that exhibits little or no toxicity. A dose may vary within this range depending upon the dosage form employed. In certain embodiments, an escalating dose may be administered.

### EXAMPLES

The following examples illustrate various aspects of the disclosure. It will be apparent to those skilled in the art that many modifications, both to materials and methods, may be practiced without departing from the scope of the disclosure.

### Example 1

Compression coated tablets containing (N,N-Diethylcarbamoyl)methyl methyl (2E)but-2-ene-1,4-dioate were made having the ingredients shown in Table 1:

TABLE 1

| | | | Quantity | Quantity |
|---|---|---|---|---|
| Component | Manufacturer | Role | (mg/tablet) | (% w/w) |
| (N,N- | XenoPort | Drug | 100.00 | 29.19 |
| Diethylcarbamoyl)methyl | (Santa Clara, CA) | substance | | |
| methyl (2E)but-2-ene- | | | | |
| 1,4-dioate | | | | |
| Hydroxypropyl Cellulose | Aquaion | Binder | 3.12 | 0.91 |
| | (Hopewell, VA) | | | |
| Hypromellose 2208 | Dow Chemical | Sustained | 9.14 | 2.67 |
| (100000 mPa · s) | (Midland, MI) | Release | | |
| | | Polymer | | |
| Silicon Dioxide | Cabot (Tuscola, IL) | Glidant | 0.23 | 0.06 |
| Magnesium Stearate | Mallinckrodt | Lubricant | 1.71 | 0.50 |
| | (St. Louis, MO) | | | |
| | | Total Core | 114.20 | 33.33 |
| Lactose Hydrate | Foremost | Filler | 157.60 | 46.00 |
| | (Rothschild, WI) | | | |
| Hypromellose 2208 | Dow Chemical | Sustained | 68.52 | 20.00 |
| (100 mPa · s) | (Midland, MI) | Release | | |
| | | Polymer | | |
| Magnesium Stearate | Mallinckrodt | Lubricant | 2.28 | 0.67 |
| | (St. Louis, MO) | | | |
| | | Total Mantle | 228.40 | 66.67 |
| | | Total Tablet | 342.60 | 100.00 |

The tablets were made according to the following steps. The core tablets were prepared using a wet granulation process. The granulation batch size was 680 g. (N,N-Diethylcarbamoyl) methyl methyl (2E)but-2-ene-1,4-dioate was passed through the Quadro Comil U5 with an 813 micron screen at 2000 rpm. Hydroxypropyl cellulose was passed through a 600 micron mesh screen. (N,N-Diethylcarbamoyl)methyl methyl (2E)but-2-ene-1,4-dioate and hydroxypropyl cellulose were granulated with purified water using a Diosna P1/6 equipped with a 4 L bowl. The wet granules were screened through an 1180 micron mesh screen and dried on trays in an oven at 30° C. for 6 hours.

The core blend batch size was 5 g. The dried granules, hydroxypropyl methyl cellulose (i.e., hypromellose 2208 having 100000 mPa·s viscosity), and the silicon dioxide were then passed through a 600 micron mesh screen, combined in a glass jar and blended on a Turbula mixer for 5 minutes. Magnesium stearate was passed through a 250 micron screen and added to the blend before blending an additional 1.5 minutes. Core tablets (114.2 mg) were compressed using a Carver Press with ¼ inch (6.35 mm) round standard concave tooling at 0.4 metric ton (MT) force. The core tablets had a final hardness of approximately 7.6 kp (˜74 Newtons).

The mantle blend was prepared using a direct compression process and a batch size of 10 g. The hypromellose 2208 (100 MPa·s viscosity) and lactose hydrate were passed through a 600 micron mesh screen, combined in a glass jar and blended on a Turbula mixer for 5 minutes. Magnesium stearate was passed through a 250 micron screen and added to the blend and blended an additional 1.5 minutes. The mantle blend was then applied to the core tablets using the Carver Press with ⅜ inch (9.53 mm) round standard concave tooling. Half the mantle blend (114.2 mg) was weighed out, added to the die, and tamped slightly to flatten. Then, the core tablet was placed into the die and pressed down gently into the mantle blend. The second half of the mantle blend (114.2 mg) was then added on top of the core tablet and the mantle was compressed using 1.5 MT force. The final compression coated tablets had a total weight of 342.6 mg with a (N,N-Diethylcarbamoyl)methyl methyl (2E)but-2-ene-1,4-dioate loading of 100 mg (29.19%). The tablets had a final hardness around 14.7 kp (˜144 Newtons).

### Example 2

Compression coated tablets containing (N,N-Diethylcarbamoyl)methyl methyl (2E)but-2-ene-1,4-dioate were made having the ingredients shown in Table 2:

TABLE 2

| | | | Quantity | Quantity |
|---|---|---|---|---|
| Component | Manufacturer | Role | (mg/tablet) | (% w/w) |
| (N,N- | XenoPort | Drug | 100.00 | 31.78 |
| Diethylcarbamoyl)methyl | (Santa Clara, CA) | substance | | |
| methyl (2E)but-2-ene- | | | | |
| 1,4-dioate | | | | |
| Hydroxypropyl Cellulose | Aqualon | Binder | 3.12 | 0.99 |
| | (Hopewell, VA) | | | |
| Silicon Dioxide | Cabot (Tuscola, IL) | Glidant | 0.21 | 0.06 |
| Magnesium Stearate | Mallinckrodt | Lubricant | 1.57 | 0.50 |
| | (St. Louis, MO) | | | |
| | | Total Core | 104.90 | 33.33 |
| Lactose Hydrate | Foremost | Filler | 144.76 | 46.00 |
| | (Rothschild, WI) | | | |
| Hypromellose 2208 | Dow Chemical | Sustained | 62.94 | 20.00 |
| (100000 mPa · s) | (Midland, MI) | Release | | |
| | | Polymer | | |
| Magnesium Stearate | Mallinckrodt | Lubricant | 2.10 | 0.67 |
| | (St. Louis, MO) | | | |
| | | Total Mantle | 209.80 | 66.67 |
| | | Total Tablet | 314.70 | 100.00 |

The tablets were made according to the following steps. The core tablets were prepared using a wet granulation process. The granulation batch size was 680 g. (N,N-Diethylcarbamoyl) methyl methyl (2E)but-2-ene-1,4-dioate was passed through the Quadro Comil U5 with an 813 micron screen at 2000 rpm. Hydroxypropyl cellulose was passed through a 600 micron mesh screen. (N,N-Diethylcarbamoyl)methyl methyl (2E)but-2-ene-1,4-dioate and hydroxypropyl cellulose were granulated with purified water using a Diosna P1/6 equipped with a 4 L bowl. The wet granules were screened through an 1180 micron mesh screen and dried on trays in an oven at 30° C. for 6 hours.

The core blend batch size was 5 g. The dried granules and the silicon dioxide were then passed through a 600 micron mesh screen, combined in a glass jar and blended on a Turbula mixer for 5 minutes. Magnesium stearate was passed through a 250 micron screen and added to the blend before blending an additional 1.5 minutes. Core tablets (104.9 mg) were compressed using a Carver Press with ¼ inch (6.35 mm) round standard concave tooling at 0.4 metric ton (MT) force. The core tablets had a final hardness of approximately 6.1 kp (˜60 Newtons).

The mantle blend was prepared using a direct compression process and a batch size of 100 g. The hydroxypropyl methyl cellulose (i.e., hypromellose 2208 having 100000 M Pa·s viscosity) and lactose hydrate were passed through a 600 micron mesh screen, combined in a 1 quart (0.95 I) V-blender and blended for 10 minutes. Magnesium stearate was passed through a 250 micron screen and added to the blend and blended an additional 4 minutes.

The mantle blend was then applied to the core tablets using the Carver Press with ⅜ inch (9.53 mm) round standard concave tooling. Half the mantle blend (104.9 mg) was weighed out, added to the die, and tamped slightly to flatten. Then, the core tablet was placed into the die and pressed down gently into the mantle blend. The second half of the mantle blend (104.9 mg) was then added on top of the core tablet and the mantle was compressed using 1.5 MT force. The final compression coated tablets had a total weight of 314.7 mg with a (N,N-Diethylcarbamoyl)methyl methyl (2E)but-2-ene-1,4-dioate loading of 100 mg (31.78%). The tablets had a final hardness around 13.1 kp (˜128 Newtons).

### Example 3

Compression coated tablets containing (N,N-Diethylcarbamoyl)methyl methyl (2E)but-2-ene-1,4-dioate were made having the ingredients shown in Table 3:

TABLE 3

| | | | Quantity | Quantity |
|---|---|---|---|---|
| Component | Manufacturer | Role | (mg/tablet) | (% w/w) |
| (N,N- | Cambridge Major | Drug | 100.0 | 27.59 |
| Diethylcarbamoyl)methyl | (Germantown, WI) | substance | | |
| methyl (2E)but-2-ene- | | | | |
| 1,4-dioate | | | | |
| Hydroxypropyl Cellulose | Aqualon | Binder | 3.1 | 0.86 |
| | (Hopewell, VA) | | | |
| Hypromellose 2208 | Dow Chemical | Sustained | 9.1 | 2.51 |
| (100000 mPa · s) | (Midland, MI) | Release | | |
| | | Polymer | | |
| Silicon Dioxide | Evonik | Glidant | 0.6 | 0.17 |
| | (Rheinfelden, | | | |
| | Germany) | | | |
| Magnesium Stearate | Mallinckrodt | Lubricant | 1.7 | 0.47 |
| | (St. Louis, MO) | | | |
| | | Total Core | 114.5 | 31.59 |
| Lactose Hydrate | Foremost | Filler | 164.8 | 45.47 |
| | (Rothschild, WI) | | | |
| Hypromellose 2208 | Dow Chemical | Sustained | 80.6 | 22.24 |
| (100 mPa · s) | (Midland, MI) | Release | | |
| | | Polymer | | |
| Magnesium Stearate | Mallinckrodt | Lubricant | 2.5 | 0.69 |
| | (St. Louis, MO) | | | |
| | | Total Mantle | 247.9 | 68.41 |
| | | Total Tablet | 362.4 | 100.00 |

The tablets were made according to the following steps. The core tablets were prepared using a wet granulation process. The granulation was performed in 2 batches at 494.88 g each. (N,N-Diethylcarbamoyl)methyl methyl (2E)but-2-ene-1,4-dioate was passed through a 1.0 mm mesh screen. Hydroxypropyl cellulose was passed through a 600 micron mesh screen. (N, N-Diethylcarbamoyl)methyl methyl (2E)but-2-ene-1,4-dioate and hydroxypropyl cellulose were combined in a 3 L bowl and mixed for 10 minutes using the Quintech granulator. The mixture was then transferred to a 2 L bowl granulated with purified water using the Quintech granulator. The wet granules were screened through a 2000 micron mesh screen and dried on trays in an oven at 30° C. for 4 hours 20 minutes. The dried granules were then passed through an 850 micron screen.

The core blend batch size was 1099.2 g. The hydroxypropyl methyl cellulose (i.e., Hypromellose 2208 having 100000 mPa·s viscosity) and the silicon dioxide were combined, passed through a 600 micron mesh screen, and added to the dry granules in a 5 L cube blender and blended for 10 minutes at 25 rpm. Magnesium stearate was passed through a 600 micron screen and added to the blend before blending an additional 4 minutes at 25 rpm. Core tablets (114.5 mg) were compressed using a Manesty F3 tablet press with 6.0 mm round concave tooling. The core tablets had a final mean hardness between 8.1 to 10.2 kp (79-100 Newtons).

The mantle blend was prepared using a direct compression process and a batch size of 5.0 kg. The hypromellose 2208 (100 MPa·s viscosity) and lactose hydrate were combined and passed through a 600 micron mesh screen, placed in and blended on the Tumblemix 18 L Bin Blender for 8.5 minutes at 30 rpm. Magnesium stearate was passed through a 600 micron screen and added to the blend and blended an additional 3.5 minutes. The mantle blend was then applied to the core tablets using a Kikusui tablet press (Kikusui Seisakusho Ltd., Kyoto, Japan) specially designed for the manufacture of compression coated tablets. Compression was completed using 9.5 mm round concave tooling and approximately 1000 kp force. The final compression coated tablets had a total weight of 362.4 mg with a (N,N-Diethylcarbamoyl)methyl methyl (2E)but-2-ene-1,4-dioate loading of 100 mg (27.59%). The compression coated tablets had a final mean hardness between 10.9 to 14.0 kp (107-137 Newtons).

### Example 4

A two-stage dissolution method was used to determine the in vitro dissolution profile of dosage forms prepared according to Examples 1, 2, and 3 in order to mimic the conditions of a dosage form as it transits the gastrointestinal tract. Thus, the dosage forms were first placed into a dissolution medium having a pH of 1.2, to mimic the conditions of the stomach, and then placed into a dissolution medium of pH 6.8, to mimic the conditions of the intestines. The dissolution vessel (USP, Type I, basket) initially contained 750 ml of 0.1 N hydrochloric acid (pH 1.2). After 2 hours of dissolution, 250 ml of 200 mM tribasic sodium phosphate was added to the vessel resulting in a pH adjustment from 1.2 to 6.8. The dissolution medium was kept at 37° C. and was agitated at 100 rpm.

For the tested dosage forms, samples of the dissolution medium were withdrawn at the indicated time points shown in the respective figures. The amount of (N,N-Diethylcarbamoyl) methyl methyl (2E)but-2-ene-1,4-dioate in the dissolution medium samples was determined by reverse phase HPLC using a C18 column and a 7 minute gradient method according to Table 4 where Mobile Phase A is water/0.1% H₃PO₄ and Mobile Phase B is water/acetonitrile/H₃PO₄ (10/90/0.1 by volume) with UV detection at 210 nm.

TABLE 4

| Time (minute) | % Mobile Phase A | % Mobile Phase B |
|---|---|---|
| 0 | 85 | 15 |
| 5 | 35 | 65 |
| 5.5 | 85 | 15 |
| 7 | 85 | 15 |

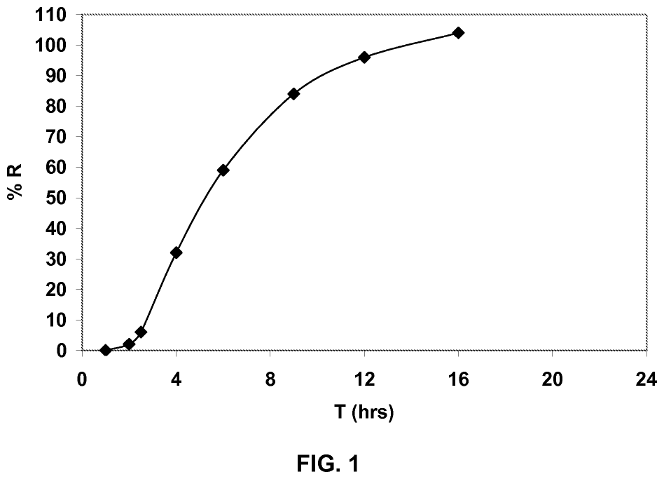

As shown in FIG. 1, for dosage forms prepared according to Example 1, drug release is delayed for approximately 2 hours, and thereafter the drug is released gradually, reaching more than 90% released at 16 hours.

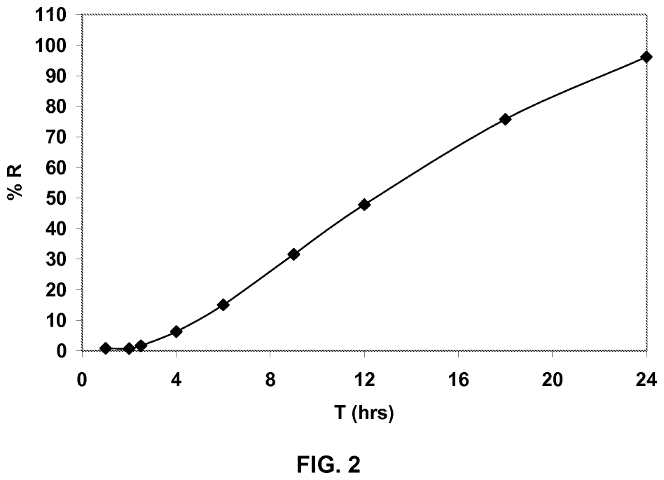

As shown in FIG. 2, for dosage forms prepared according to Example 2, drug release is delayed for approximately 2 hours, followed by near zero order release, reaching more than 90% released at 24 hours.

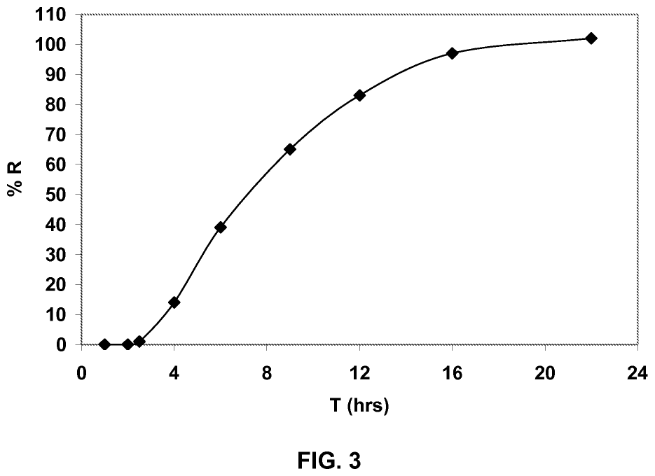

As shown in FIG. 3, for dosage forms prepared according to Example 3, drug release is delayed for approximately 2 hours, and thereafter the drug is released gradually, reaching more than 90% released at 16 hours.

### Example 5

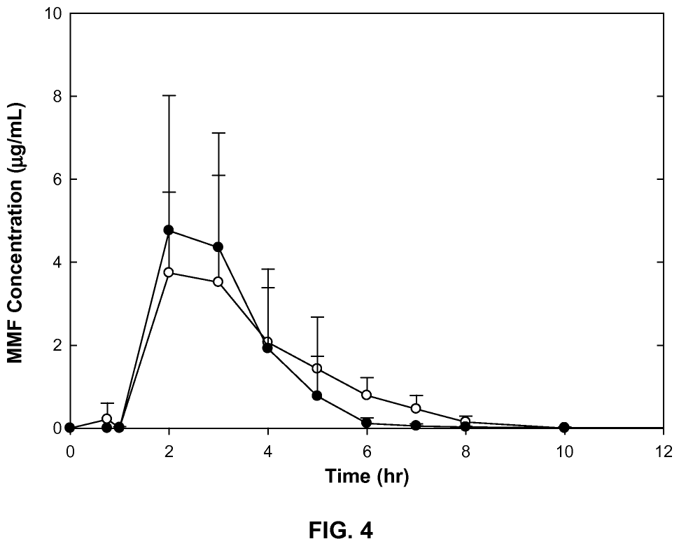

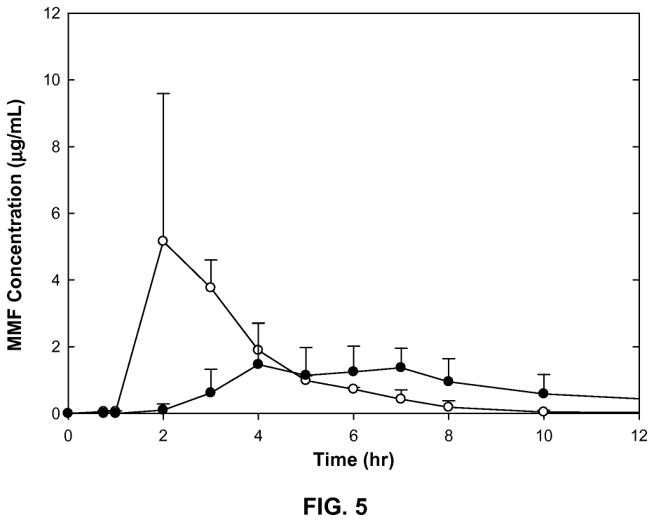

The concentration±1 SD of MHF in the blood of Cynomologous monkeys following oral dosing of delayed release enteric coated tablets prepared according to Examples 1 and 2 is shown in FIGS. 4 and 5. In these Figures, the MHF concentrations following dosing with the Example 1 tablets are shown with o-o symbols and the MHF concentrations following dosing with the Example 2 tablets are shown with ●-● symbols. The data in FIG. 4 is from animals dosed in a fasted state and the data in FIG. 5 is from animals dosed in a fed state.

### Administration Protocol

Tablets prepared according to Examples 1 and 2 (100 mg (N,N-Diethylcarbamoyl)methyl methyl (2E)but-2-ene-1,4-dioate per tablet) were administered by oral dosing to groups of four adult male Cynomologous (Macaca fascicularis) monkeys (each monkey weighed about 4 to 5 kg). Each monkey was administered two tablets in either a fasted state or a fed state. All animals were fasted overnight before the study. For the fed leg, animals were administered blended food via oral gavage in the morning 30 minutes prior to administration of each test formulation. For the fasted leg, the animals remained fasted for 4 hours post dosing. Blood samples (1.0 mL) were obtained from all animals via the femoral vein at pre-dose and intervals over 24 hours after oral dosing. Blood was collected in pre-chilled

K2EDTA, quenched with acetonitrile and stored at −50° C. to −90° C.until analyzed. There was a minimum 7 day wash out period between dosing sessions.

### Sample Preparation for Absorbed Drug

300 μL of acetonitrile was added to 1.5 mL Eppendorf tubes for the preparation of samples and standards.

Sample Preparation: Blood was collected at different time points and immediately 100 μL of blood was added into Eppendorf tubes containing 300 μL of methanol and mixed by vortexing.

Standard Preparation: One hundred μL of blood was added to 290 μL of acetonitrile in Eppendorf tubes. 10 μL of MMF standard solution (0.2, 0.5, 1, 2.5, 5, 10, 25, 50 and 100 μg/mL) was added to each tube to make up the final calibration standards (0.02, 0.05, 0.1, 0.25, 0.5, 1, 2.5, 5 and 10 μg/mL).

A 150 μL aliquot of supernatant from quenched blood standards, QCs and samples was transferred to a 96-well plate and 20 μL of the internal standard solution was added to each well, the plate was capped and vortexed well. The supernatant was injected onto the API 4000 LC/MS/MS system for analysis

### LC/MS/MS Analysis

The concentration of MMF in monkey blood was determined using an API 4000 LC/MS/MS instrument equipped with Agilent Binary pump and autosampler. The column was a Luna C8 (2) 4.6×150 mm, 5μ column operating at 2 to 8° C. temperature. The mobile phases were (A) 0.1% formic acid in water, and (B) 0.1% formic acid in acetonitrile. The gradient condition was: 2% B for 1 min, increasing to 95% B in 3.5 min and maintained for 2 min, then decreasing to 2% B in 5.6 min and maintained for 2.3 min. 30 μL of sample was injected into the column. A Turbo-Ion Spray source was used, and was detected in negative ion mode for the MRM transition of 128.95/84.8. Peaks were integrated using Analyst 1.5 quantitation software.

### Example 6

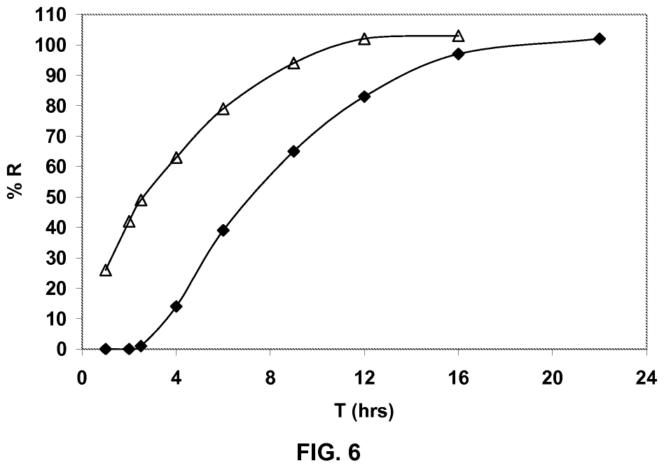

To demonstrate the effect of the compression coating on the in vitro dissolution profile from tablets made according to Example 3, the dissolution profile from the cores of the compression coated tablets of Example 3 (i.e., the intermediate product before application of the mantle) was tested according to the method described in Example 4 and compared to the dissolution profile from the finished compression coated tablets of Example 3. FIG. 6 shows that the compression coating provides a 2 hour delay before drug release as shown by comparing the profiles of the cores (Δ-Δ symbols) and the compression coated tablets (⋄-⋄ symbols).

### Example 7

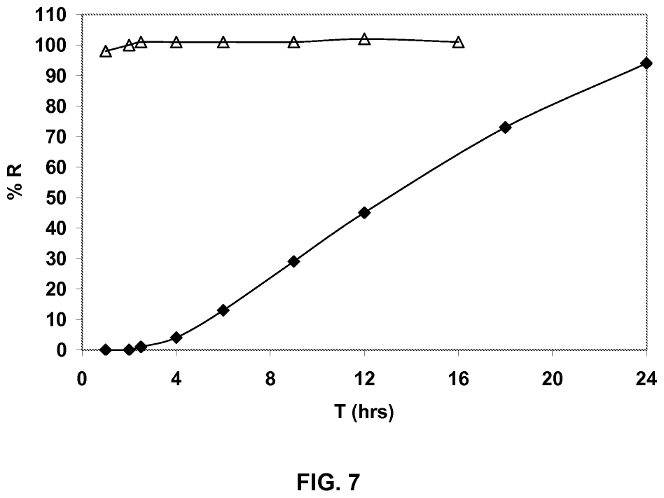

To demonstrate the effect of the compression coating on the in vitro dissolution profile from tablets made according to Example 2, the dissolution profile from the cores of the compression coated tablets of Example 2 (i.e., the intermediate product before application of the mantle) was tested according to the method described in Example 4 and compared to the dissolution profile from the finished compression coated tablets of Example 2. FIG. 7 shows that the compression coating provides a 2 hour delay and a near zero order release profile as shown by comparing the profiles of the cores (Δ-Δ symbols) and the compression coated tablets (⋄-⋄ symbols).

### Example 8

To demonstrate the effect of increasing the percentage of sustained release polymer in the core on the in vitro dissolution profile, two different tablet formulations were made according to the procedure outlined in Example 1, but with significantly differing levels of hypromellose 2208 (100000 MPa·s viscosity) in the core, i.e., compared to the Example 1 tablets. Thus, the Example 1 tablets contained 8 wt % HPMC while the two Example 8 tablets contained 5 wt % and 10 wt % HPMC, respectively. The tablet formulations, including the Example 1 tablet formulation for reference, are shown in Table 5.

TABLE 5

| | Quantity | Quantity | Quantity | Quantity | Quantity | Quantity |
|---|---|---|---|---|---|---|
| | (mg/tablet) | (% w/w) | (mg/tablet) | (% w/w) | (mg/tablet) | (% w/w) |
| Component | Example 1 | | Example 8a | | Example 8b | |
| (N,N- | 100.00 | 29.19 | 100.00 | 30.17 | 100.00 | 28.55 |
| Diethylcarbamoyl)methyl | | | | | | |
| methyl (2E)but-2- | | | | | | |
| ene-1,4-dioate | | | | | | |
| Hydroxypropyl | 3.12 | 0.91 | 3.10 | 0.93 | 3.10 | 0.88 |
| Cellulose | | | | | | |
| Hypromellose 2208 | 9.14 | 2.67 | 5.52 | 1.66 | 11.67 | 3.33 |
| (100000 mPa · s) | | | | | | |
| Silicon Dioxide | 0.23 | 0.06 | 0.22 | 0.07 | 0.23 | 0.07 |
| Magnesium Stearate | 1.71 | 0.50 | 1.66 | 0.50 | 1.75 | 0.50 |
| Total Core | 114.20 | 33.33 | 110.50 | 33.33 | 116.75 | 33.33 |
| Lactose Hydrate | 157.60 | 46.00 | 152.49 | 46.00 | 161.12 | 46.00 |
| Hypromellose 2208 | 68.52 | 20.00 | 66.30 | 20.00 | 70.05 | 20.00 |
| (100 mPa · s) | | | | | | |
| Magnesium Stearate | 2.28 | 0.67 | 2.21 | 0.67 | 2.33 | 0.67 |
| Total Mantle | 228.40 | 66.67 | 221.00 | 66.67 | 233.50 | 66.67 |
| Total Tablet | 342.60 | 100.00 | 331.50 | 100.00 | 350.25 | 100.00 |

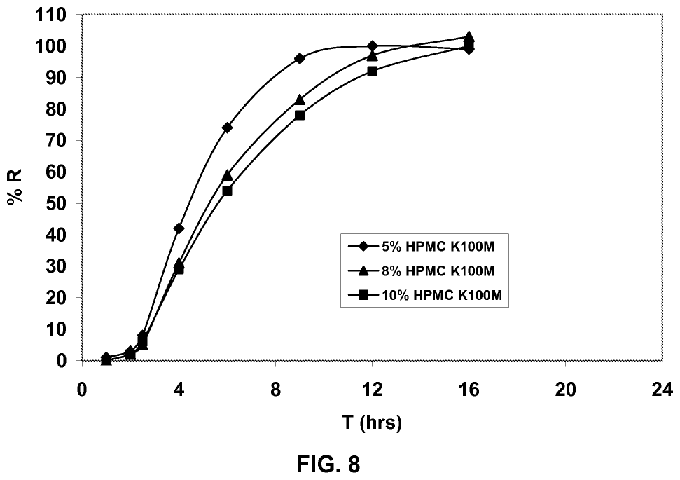

The dissolution profiles from the three compression coated tablets were measured according to the method described in Example 4. FIG. 8 shows that the MHF prodrug release rate slows with increasing percentage of hypromellose 2208 (100000 mPa·s) in the core, but the initial delay before the start of prodrug release stays the same at approximately 2 hours, likely due to the unchanged mantle layer.

### Example 9

To demonstrate the effect of increasing the viscosity of sustained release polymer in the mantle on the in vitro dissolution profile, tablets were made with hypromellose 2208 of different viscosities in the mantle: Example 9a (100 mPa·s), Example 9b (4000 mPa·s), and Example 9c (a combination of 100 mPa·s and 4000 mPa·s to give an effective viscosity of ˜2000 mPa·s). The formulation details are shown in Table 6.

TABLE 6

| | Quantity | Quantity | Quantity | Quantity | Quantity | Quantity |
|---|---|---|---|---|---|---|
| | (mg/tablet) | (% w/w) | (mg/tablet) | (% w/w) | (mg/tablet) | (% w/w) |
| Component | Example 9a | | Example 9b | | Example 9c | |
| (N,N- | 200.00 | 32.00 | 200.00 | 32.00 | 200.00 | 32.00 |
| Diethylcarbamoyl)methyl | | | | | | |
| methyl (2E)but-2- | | | | | | |
| ene-1,4-dioate | | | | | | |
| Hydroxypropyl | 6.20 | 1.00 | 6.20 | 1.00 | 6.20 | 1.00 |
| Cellulose | | | | | | |
| Magnesium Stearate | 2.10 | 0.30 | 2.10 | 0.30 | 2.10 | 0.30 |
| Total Core | 208.30 | 33.30 | 208.30 | 33.30 | 208.30 | 33.30 |
| Lactose Hydrate | 308.30 | 49.30 | 308.30 | 49.30 | 308.30 | 49.30 |
| Hypromellose 2208 | 104.10 | 16.70 | 0.00 | 0.00 | 52.05 | 8.35 |
| (100 mPa · s) | | | | | | |
| Hypromellose 2208 | 0.00 | 0.00 | 104.10 | 16.70 | 52.05 | 8.35 |
| (4000 mPa · s) | | | | | | |
| Magnesium Stearate | 4.20 | 0.70 | 4.20 | 0.70 | 4.20 | 0.70 |
| Total Mantle | 416.60 | 66.7 | 221.00 | 66.70 | 233.50 | 66.70 |
| Total Tablet | 624.90 | 100.0 | 331.50 | 100.00 | 350.25 | 100.00 |

The tablets were made according to the following steps. The core tablets were prepared using a wet granulation process. The granulation batch size was 170 g. (N,N-Diethylcarbamoyl) methyl methyl (2E)but-2-ene-1,4-dioate was passed through the Quadro Comil U5 with an 813 micron screen at 2000 rpm. Hydroxypropyl cellulose was passed through a 500 micron mesh screen. (N,N-Diethylcarbamoyl)methyl methyl (2E)but-2-ene-1,4-dioate and hydroxypropyl cellulose were granulated with purified water using a Diosna P1/6 equipped with a 1 L bowl. The wet granules were screened through an 1180 micron mesh screen and dried on trays in an oven at 30° C. for 3 hours 48 minutes.

The core blend batch size was 20.0 g. The dried granules and magnesium stearate were combined in a glass bottle and blended on a Turbula mixer for 2 minutes. Core tablets (208.3 mg) were compressed using a Manesty FlexiTab single station tablet press with 5/16 inch (7.9 mm) round standard concave tooling at forces ranging from 9.9 to 14.0 kN. The core tablets had a final mean hardness of 8.4 kp (˜82 Newtons).

The mantle blend was prepared using a direct compression process and a batch size of either 10 g (Examples 9a and 9c) or 20 g (Example 9b). The hypromellose 2208 and lactose hydrate were passed through a 600 micron mesh screen, combined in a glass bottle and blended on a Turbula mixer for either 10 (Example 9b), 6 (Example 9a), or 5 (Example 9c) minutes. In each case, magnesium stearate was passed through a 250 micron screen and added to the blend and blended an additional 1.5 minutes. The mantle blend was then applied to the core tablets using the Carver Press with 7/16 inch (11.1 mm) round standard concave tooling. Half the mantle blend (208.3 mg) was weighed out, added to the die, and tamped slightly to flatten. Then, the core tablet was placed into the die and pressed down gently into the mantle blend. The second half of the mantle blend (208.3 mg) was then added on top of the core tablet and the mantle was compressed using 2.0 metric ton (MT) force. The final compression coated tablets had a total weight of 624.9 mg with a (N,N-Diethylcarbamoyl) methyl methyl (2E)but-2-ene-1,4-dioate loading of 200 mg (32.00%). The tablets had a final hardness of about 18.3 to 19.5 kp (˜179 to 191 Newtons).

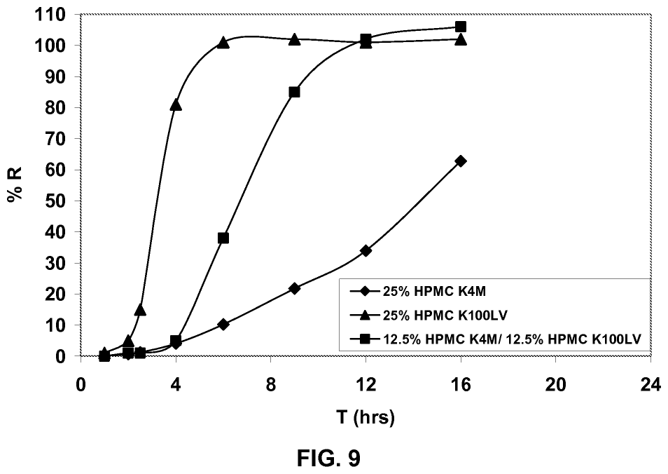

The dissolution profiles from the three compression coated tablets were measured according to the method described in Example 4. FIG. 9 shows that the MHF prodrug release rate slows with increasing hypromellose viscosity and the delay time increases with increasing hypromellose viscosity.

### Example 10

To demonstrate the effect of increasing the percentage of hypromellose 2208 (100 mPa·s viscosity) in the mantle on the in vitro dissolution profile, tablets were made according to the procedure outlined in Example 1, but with 5 wt % hypromellose 2208 (100000 mPa·s) in the core and different levels of hypromellose 2208 (100 MPa·s) in the mantle: Example 8a (30% hypromellose in mantle) and Example 10 (40% hypromellose in mantle). The tablet formulations, including the Example 1 and Example 8a tablet formulations for reference, are shown in Table 7.

TABLE 7

| | Quantity | Quantity | Quantity | Quantity | Quantity | Quantity |
|---|---|---|---|---|---|---|
| | (mg/tablet) | (% w/w) | (mg/tablet) | (% w/w) | (mg/tablet) | (% w/w) |
| Component | Example 1 | | Example 8a | | Example 10 | |
| (N,N- | 100.00 | 29.19 | 100.00 | 30.17 | 100.00 | 30.17 |
| Diethylcarbamoyl)methyl | | | | | | |
| methyl (2E)but-2- | | | | | | |
| ene-1,4-dioate | | | | | | |
| Hydroxypropyl | 3.12 | 0.91 | 3.10 | 0.93 | 3.10 | 0.93 |
| Cellulose | | | | | | |
| Hypromellose 2208 | 9.14 | 2.67 | 5.52 | 1.66 | 5.52 | 1.66 |
| (100000 mPa · s) | | | | | | |
| Silicon Dioxide | 0.23 | 0.06 | 0.22 | 0.07 | 0.22 | 0.07 |
| Magnesium Stearate | 1.71 | 0.50 | 1.66 | 0.50 | 1.66 | 0.50 |
| Total Core | 114.20 | 33.33 | 110.50 | 33.33 | 110.50 | 33.33 |
| Lactose Hydrate | 157.60 | 46.00 | 152.49 | 46.00 | 130.39 | 39.33 |
| Hypromellose 2208 | 68.52 | 20.00 | 66.30 | 20.00 | 88.40 | 26.67 |
| (100 mPa · s) | | | | | | |
| Magnesium Stearate | 2.28 | 0.67 | 2.21 | 0.67 | 2.21 | 0.67 |
| Total Mantle | 228.40 | 66.67 | 221.00 | 66.67 | 221.00 | 66.67 |
| Total Tablet | 342.60 | 100.00 | 331.50 | 100.00 | 331.50 | 100.00 |

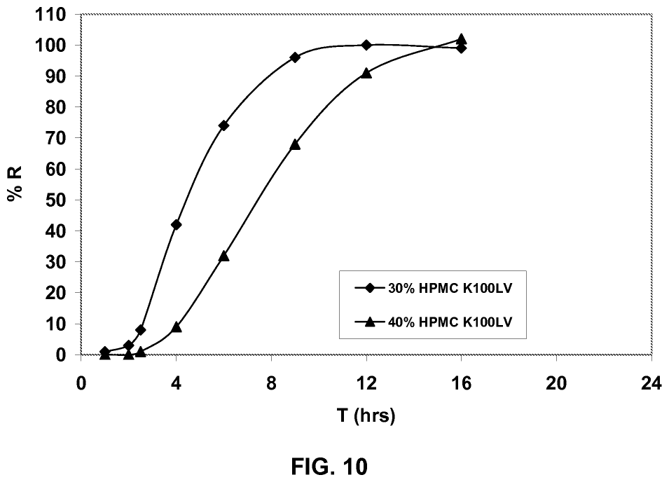

The dissolution profiles from the three compression coated tablets were measured according to the method described in Example 4. FIG. 10 shows that the rate of MHS prodrug release slows and the delay to drug release is increased with increasing percentage of hypromellose 2208 (100 mPa·s) in the mantle.

### Example 11

To demonstrate the effect of increasing the percentage of hypromellose 2208 (100000 mPa·s) in the mantle on the in vitro dissolution profile, tablets were made with two different levels of hypromellose 2208 (100000 mPa·s) in the mantle: Example 11a (20%) and Example 11b (30%). The tablet formulations are shown in Table 8.

TABLE 8

| | Quantity | Quantity | Quantity | Quantity |
|---|---|---|---|---|
| | (mg/tablet) | (% w/w) | (mg/tablet) | (% w/w) |
| Component | Example 11a | | Example 11b | |
| (N,N- | 100.00 | 38.37 | 100.00 | 38.37 |
| Diethylcarbamoyl)methyl | | | | |
| methyl (2E)but-2-ene- | | | | |
| 1,4-dioate | | | | |
| Hydroxypropyl Cellulose | 3.06 | 1.17 | 3.06 | 1.17 |
| Silicon Dioxide | 0.10 | 0.04 | 0.10 | 0.04 |
| Magnesium Stearate | 1.04 | 0.40 | 1.04 | 0.40 |
| Total Core | 104.20 | 40.00 | 104.20 | 40.00 |
| Lactose Hydrate | 107.92 | 41.41 | 92.28 | 35.40 |
| Hypromellose 2208 | 46.92 | 18.01 | 62.56 | 24.00 |
| (100000 mPa · s) | | | | |
| Magnesium Stearate | 1.56 | 0.60 | 1.56 | 0.60 |
| Total Mantle | 156.40 | 60.00 | 156.40 | 66.70 |
| Total Tablet | 260.60 | 100.00 | 260.60 | 100.00 |

The tablets were made according to the following steps. The core tablets were prepared using a wet granulation process. The granulation batch size was 680 g. (N,N-Diethylcarbamoyl)methyl methyl (2E)but-2-ene-1,4-dioate was passed through the Quadro Comil U5 with an 813 micron screen at 2000 rpm. Hydroxypropyl cellulose was passed through a 600 micron mesh screen. (N,N-Diethylcarbamoyl)methyl methyl (2E)but-2-ene-1,4-dioate and hydroxypropyl cellulose were granulated with purified water using a Diosna P1/6 equipped with a 4 L bowl. The wet granules were screened through an 1180 micron mesh screen and dried on trays in an oven at 30° C. for 6 hours.

The core blend batch size was 30.0 g. The dried granules and the silicon dioxide were then passed through a 600 micron mesh screen, combined in a glass jar and blended in a Turbula mixer for 2 minutes. Magnesium stearate was passed through a 250 micron screen and added to the blend before blending an additional 1.5 minutes. Core tablets (104.2 mg) were compressed using a Manesty FlexiTab single station tablet press with ¼ inch (6.35 mm) round standard concave tooling at approximately 3 kN force. The core tablets had a final hardness of 6.2 to 7.0 kp (about 61 to 69 Newtons).

The mantle blend was prepared using a direct compression process and a batch size of 10 g. The hypromellose 2208 (100000 MPa·s) and lactose hydrate were passed through a 600 micron mesh screen, combined in a glass bottle and blended for 5 minutes on a Turbula mixer. Magnesium stearate was passed through a 250 micron screen and added to the blend and blended an additional 1.5 minutes. The mantle blend was then applied to the core tablets using the Carver Press with 5/16 inch (7.94 mm) round standard concave tooling. Half the mantle blend (78.2 mg) was weighed out, added to the die, and tamped slightly to flatten. Then, the core tablet was placed into the die and pressed down gently into the mantle blend. The second half of the mantle blend (78.2 mg) was then added on top of the core tablet and the mantle was compressed using 1.1 metric ton (MT) force. The final compression coated tablets had a total weight of 260.6 mg with a (N,N-Diethylcarbamoyl)methyl methyl (2E)but-2-ene-1,4-dioate loading of 100 mg (38.37%). The tablets had a final hardness ranging from 13.1 to 14.0 kp (about 128 to 137 Newtons).

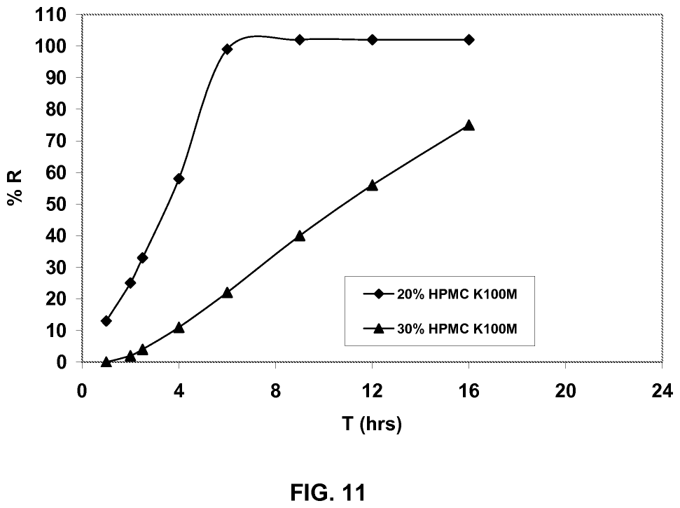

The dissolution profiles from the two compression coated tablets were measured according to the method described in Example 4. FIG. 11 shows that the release slows with increasing percentage of hypromellose 2208 (100000 mPas) in the mantle.

### Example 12

A stability study was conducted on the (N, N-Diethylcarbamoyl)methyl methyl (2E)but-2-ene-1,4-dioate-containing compression coated tablets, 100 mg at 5° C., 25° C./60% RH, 30° C./65% RH, and 40° C./75% RH. The tablets used in the stability study had the composition outlined in Table 9.

TABLE 9

| | Quantity | Quantity |
|---|---|---|
| | (mg/tablet) | (% w/w) |
| Component | Example 12 | |
| (N,N- | 99.74 | 27.63 |
| Diethylcarbamoyl)methyl | | |
| methyl (2E)but-2-ene- | | |
| 1,4-dioate | | |
| Hydroxypropyl Cellulose | 3.08 | 0.85 |
| Hypromellose 2208 | 9.07 | 2.51 |
| (100000 mPa s) | | |
| Silicon Dioxide | 0.51 | 0.14 |
| Magnesium Stearate | 1.70 | 0.47 |
| Total Core | 114.10 | 31.60 |
| Lactose Hydrate | 164.25 | 45.49 |
| Hypromellose 2208 | 80.28 | 22.23 |
| (100000 mPa · s) | | |
| Magnesium Stearate | 2.47 | 0.68 |
| Total Mantle | 247.00 | 68.40 |
| Total Tablet | 361.10 | 100.00 |

The tablets were made according to the following steps. The core tablets were prepared using a wet granulation process. The granulation was performed in 2 batches at 494.88 g each. (N,N-Diethylcarbamoyl)methyl methyl (2E)but-2-ene-1,4-dioate was passed through a 1.0 mm mesh screen. Hydroxypropyl cellulose was passed through a 600 micron mesh screen. (N,N-Diethylcarbamoyl)methyl methyl (2E)but-2-ene-1,4-dioate and hydroxypropyl cellulose were combined in a 3 L bowl and mixed for 2 minutes using the Quintech granulator. The mixture was then transferred to a 2 L bowl granulated with purified water using the Quintech granulator. The wet granules were screened through a 2000 micron mesh screen and dried in a Glatt Fluid Bed Drier at 40° C. for 15 minutes, 39 sec. The dried granules were then passed through an 800 micron screen.

The core blend batch size was 1095.36 g. The hypromellose 2208 (100000 mPa·s viscosity) and the silicon dioxide were combined, passed through a 600 micron mesh screen, and added to the dry granules in a 5 L cube blender and blended for 10 minutes at 25 rpm. Magnesium stearate was passed through a 600 micron screen and added to the blend before blending an additional 4 minutes at 25 rpm. Core tablets (114.1 mg) were compressed using a Manesty F3 tablet press with 6.0 mm round concave tooling. The core tablets had a final mean hardness of 8.6 kp (about 84 Newtons).

The mantle blend was prepared using a direct compression process and a batch size of 5.0 kg. The hypromellose 2208 (100 MPa·s viscosity) and lactose hydrate were combined and passed through a 600 micron mesh screen, placed in an 18 L Bin and blended on the Tumblemix 18 L Bin Blender for 8.5 minutes at 30 rpm. Magnesium stearate was passed through a 600 micron screen and added to the blend and blended an additional 3.5 minutes. The mantle blend was then applied to the core tablets using a Kikusui tablet press specially designed for the manufacture of compression coated tablets. Compression was completed using 9.5 mm round concave tooling and approximately 1200 kp force. The final compression coated tablets had a total weight of 361.1 mg with a (N, N-Diethylcarbamoyl)methyl methyl (2E)but-2-ene-1,4-dioate loading of 100 mg (27.63%). The compression coated tablets had a final mean hardness of 15.3 kp (about 150 Newtons).

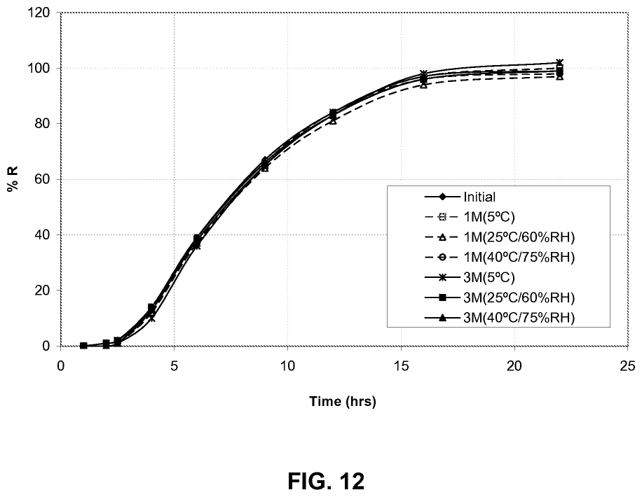

The final tablets were packaged for stability testing. The packaging configuration was thirty tablets in a 0.02 inch (0.5 mm) thick, 60 cm³ HDPE bottle with child-resistant screw cap and foil induction seal, containing a 2 g silica gel canister. The packaged tablets were placed on stability according to the protocol outlined in Table 10. The stability results for the appearance, assay/impurity, and water content are presented in Table 11. The stability results for the dissolution are presented in FIG. 12.

TABLE 10

| Storage | Stability Schedule | | | | | |
|---|---|---|---|---|---|---|
| Condition | TO | 1 | 3 | 6 | 9 | 12 |
| 5° C. | X | X | X | X | X | X |
| 25° C./60% RH | | X | X | X | X | X |
| 30° C./65% RH | | | | (X) | X | X |
| 40° C./75% RH | | X | X | X | | |

Testing included:
D = 2-stage dissolution (pH 1.2/6.8), n = 6
A = Assay/impurity and Appearance, n = 5

TABLE 11

| | | | | Total |
|---|---|---|---|---|
| Storage | Time | | Assay | Degradants |
| Condition | (month) | Appearance | (% w/w) | (% w/w) |
| Initial | 0 | White round | 99.0 | ND |
| | | tablets | | |
| 5° C. | 1 | conforms | 102.5 | ND |
| | 3 | conforms | 99.4 | ND |
| 25° C./60% RH | 1 | conforms | 103.5 | ND |
| | 3 | conforms | 101.2 | ND |
| 40° C./75% RH | 1 | conforms | 101.1 | ND |
| | 3 | conforms | 99.2 | ND |

ND = not detected

### Example 13

A randomized, double-blind crossover, food effect, single-dose study of the safety, tolerability, and pharmacokinetics of an oral dosage form of (N,N-Diethylcarbamoyl)methyl methyl (2E)but-2-ene-1,4-dioate in healthy adult subjects was conducted. Twelve healthy adult volunteers (males and females) participated in the study. All twelve subjects received 15 a dosage form of Example 3, once in a fed condition and once in a fasted condition, with a two-week washout between treatments. The fasted dosing was achieved by dosing the subject following an overnight fast while the fed dosing was achieved by dosing the subject after consuming a high fat-content breakfast. The dosage form contained 100 mg of (N,N-Diethylcarbamoyl)methyl methyl (2E)but-2-ene-1,4-dioate (54 mg equivalents of methyl hydrogen fumarate).

Blood samples were collected from all subjects prior to dosing, and at 0.5, 1, 1.5, 2, 3, 4, 5, 6, 7, 8, 10, 12, 16, 24, 30, 36, 48, 60, 72, 84, 96, 108 and 120 hours after dosing. Urine samples were collected from all subjects prior to dosing, and complete urine output was obtained at the 0-4, 4-8, 8-12, 12-24, 24-36, 36-48, 48-72, 72-96 and 96-120 hour intervals after dosing. Blood samples were quenched immediately with acetonitrile and frozen. Sample aliquots were prepared for analysis of (i) methyl hydrogen fumarate, (ii) (N,N-Diethylcarbamoyl) methyl methyl (2E)but-2-ene-1,4-dioate, (iii) N,N diethyl-2-hydroxy acetamide and (iv) (2S,3S,4S,5R,6R)-6-[(N,N-diethylcarbamoyl)methoxy]-3,4,5-trihydroxy-2H-3,4,5,6-tetrahydropyran-2-carboxylic acid, the latter two being other potential metaboliltes of (N,N-Diethylcarbamoyl)methyl methyl (2E)but-2-ene-1,4-dioate, using sensitive and specific LC/MS/MS methods.

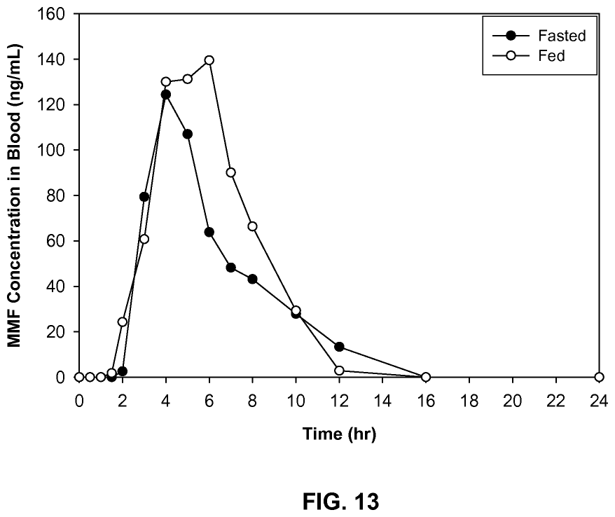

The plasma concentration of MMF following oral dosing of the formulation prepared according to Example 3 to fasted and fed healthy adult patients is shown in FIG. 13. Table 12 shows the preliminary mean (SD) pharmacokinetic data for (N,N-diethylcarbamoyl)methyl methyl (2E)but-2-ene-1,4-dioate in fed and fasted patients.

TABLE 12

| | | Cₘₐₓ | AUC_{inf} |
|---|---|---|---|
| N | Food | (ng/mL) | (ng · hr/mL) |
| 12 | Fasted | 143 | 625 |
| | | (61.1) | (216) |
| 12 | Fed | 217 | 750 |
| | | (88.5) | (242) |

MMF release from the formulation was sustained and minimally affected by food. The formulation produced mean (SD) maximum MMF concentrations (Cmax) 143 (61) ng/mL fasted and 217 (89) ng/ml fed. MMF AUC was 625 (216) ng·h/mL fasted and 750 (242) ng·h/mL fed. Promoiety was cleared from blood with a half life around 3 hours. (N,N-Diethylcarbamoyl) methyl methyl (2E)but-2-ene-1,4-dioate was well tolerated during the trial. All 12 subjects completed the dosing period. All adverse events were mild. Adverse events that were reported in more than one subject and that were more frequently for (N,N-Diethylcarbamoyl)methyl methyl (2E)but-2-ene-1,4-dioate than for placebo were flushing and feeling hot. A comparison of these adverse events to placebo is shown in Table 13.

TABLE 13

| | Flushing | | Feeling Hot | |
|---|---|---|---|---|
| | Fasted | Fed | Fasted | Fed |
| Placebo | 0 | 1 | 0 | 0 |
| Formulation | 0 | 1 | 0 | 0 |

### Example 14

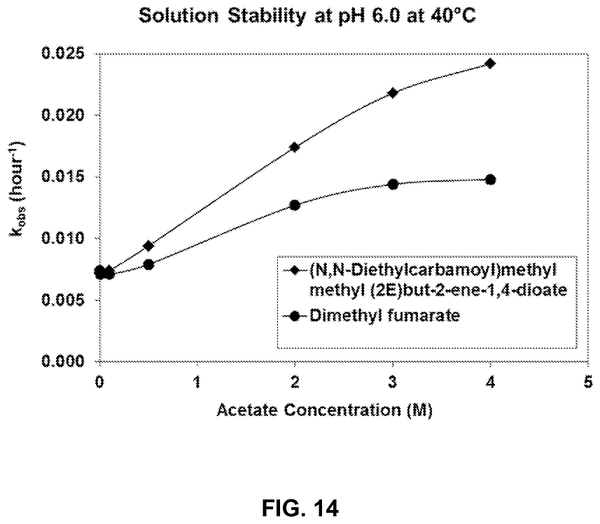

This example studied the degradation of MHF prodrugs (N,N-Diethylcarbamoyl)methyl methyl (2E)but-2-ene-1,4-dioate and DMF in the presence of varying quantities of acetic acid. Each prodrug was placed in a pH 6.0 phosphate buffer with multiple concentrations of sodium acetate (0.0 M, 0.1 M, 0.5 M, 2.0 M, 3.0 M, and 4.0 M) at 40° C. The presence of prodrug was measured over time up to 42 hours. The rate of prodrug degradation can be expressed according to the following formula: In (A)=In (A₀)−K_{obs}●t, wherein A is the prodrug concentration, A₀ is the prodrug concentration at time zero and K_{obs} is the observed slope of the curve plotting In (A) versus time (t). Thus, the higher the K_{obs}, the more quickly the prodrug is degrading. Thus, K_{obs} is a measure of prodrug stability, with prodrugs having a lower K_{obs} being more stable than prodrugs having a higher K_{obs}. The K_{obs} for (N,N-Diethylcarbamoyl)methyl methyl (2E)but-2-ene-1,4-dioate and DMF are plotted as a function of acetate concentration in FIG. 14.

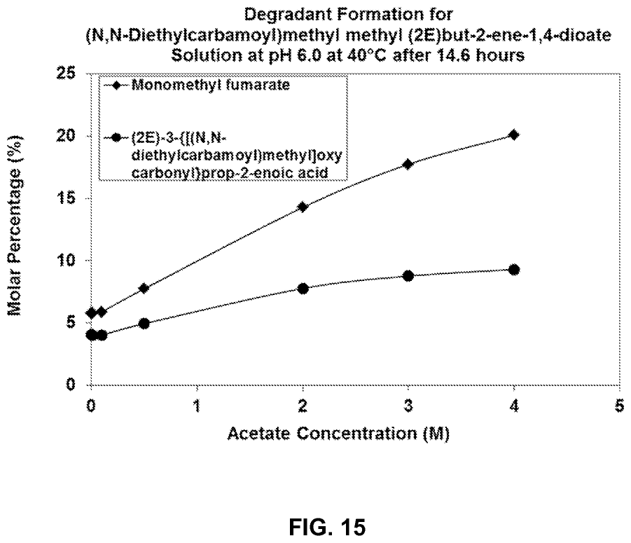

The amounts of the two primary degradation products for prodrug (N,N-Diethylcarbamoyl)methyl methyl (2E)but-2-ene-1,4-dioate were measured after a 14.6 hour exposure to acetate solutions of varying concentrations, all at pH 6.0 and 40° C. The data are shown in FIG. 15, Thus, FIG. 15 depicts the amount of each of the two primary degradation products of (N,N-Diethylcarbamoyl)methyl methyl (2E)but-2-ene-1,4-dioate as a mole % of the initial amount of prodrug, at the varying acetate concentrations.

The degradation rates of each of (N,N-Diethylcarbamoyl)methyl methyl (2E)but-2-ene-1,4-dioate and DMF increased with increasing concentrations of acetate. The effect was more pronounced for the (N,N-Diethylcarbamoyl)methyl methyl (2E)but-2-ene-1,4-dioate prodrug than for DMF. For (N, N-Diethylcarbamoyl)methyl methyl (2E)but-2-ene-1,4-dioate, the formation rates of both primary degradation products increased with increasing acetate concentration. This is consistent with the more pronounced effect of acetate seen with (N,N-Diethylcarbamoyl)methyl methyl (2E)but-2-ene-1,4-dioate compared to DMF.

Without wishing to be limited to a specific mechanism or mode of action, increased carboxyl concentration independent of pH causes increased degradation of the MHF prodrugs. It is believed that selection of pharmaceutical excipients in the core, and compression coating layer components, that are substantially free of carboxylic acid moieties reduces the degradation of the MHF prodrugs.

### Example 15

Compression coated tablets containing dimethyl fumarate were made having the ingredients shown in Table 14:

TABLE 14

| | | | Quantity | Quantity |
|---|---|---|---|---|
| Component | Manufacturer | Role | (mg/tablet) | (% w/w) |
| Dimethyl | TCI | Drug | 120.00 | 28.96 |
| Fumarate | (Portland, OR) | substance | | |
| Hydroxypropyl | Ashland | Binder | 3.63 | 0.88 |
| Cellulose | (Wilmington, DE) | | | |
| Hypromellose | Dow Chemical | Sustained | 9.41 | 2.27 |
| 2208 | (Midland, MI) | Release | | |
| (100000 mPa · s) | | Polymer | | |
| Silicon | Degussa | Glidant | 0.67 | 0.16 |
| Dioxide | (Parsippany, NJ) | | | |
| Magnesium | Mallinckrodt | Lubricant | 0.67 | 0.16 |
| Stearate | (St. Louis, MO) | | | |
| | | Total Core | 134.38 | 32.43 |
| Lactose | Foremost | Filler | 186.20 | 44.93 |
| Hydrate | (Rothschild, WI) | | | |
| Hypromellose | Dow Chemical | Sustained | 91.00 | 21.96 |
| 2208 | (Midland, MI) | Release | | |
| (100 mPa · s) | | Polymer | | |
| Magnesium | Mallinckrodt | Lubricant | 2.80 | 0.68 |
| Stearate | (St. Louis, MO) | | | |
| | | Total Mantle | 280.00 | 67.57 |
| | | Total Tablet | 414.38 | 100.00 |

The tablets were made according to the following steps. The core tablets were prepared using a direct compression process and a batch size of 30 g. The dimethyl fumarate was passed through a 180 micron mesh screen and the hydroxypropyl cellulose, hypromellose 2208 (100000 MPa·s viscosity), and silicon dioxide were passed through a 600 micron mesh screen, combined in a glass jar and blended on a Turbula mixer for 5 minutes. Magnesium stearate was passed through a 600 micron screen and added to the blend and blended an additional 1.5 minutes. Core tablets (134.4 mg) were compressed using a Carver Press with 6.00 mm round standard concave tooling at 0.3 metric ton (MT) force.

The mantle blend was prepared using a direct blending process and a batch size of 5.0 kg. The hypromellose 2208 (100 MPa·s viscosity) and lactose hydrate were combined and passed through a 600 micron mesh screen, placed in and blended on the Tumblemix 18 L Bin Blender for 8.5 minutes at 30 rpm. Magnesium stearate was passed through a 600 micron screen and added to the blend and blended an additional 3.5 minutes. The mantle blend was then applied to the core tablets using the Carver Press with 9.50 mm round standard concave tooling. Half the mantle blend (140.0 mg) was weighed out, added to the die, and tamped slightly to flatten. Then, the core tablet was placed into the die and pressed down gently into the mantle blend. The second half of the mantle blend (140.0 mg) was then added on top of the core tablet and the mantle was compressed using 1.6 MT force. The final compression coated tablets had a total weight of 414.4 mg with a dimethyl fumarate loading of 120 mg (28.96%). The (axial x radial) dimensions of the compression coated tablet were 5.92×9.54 mm. The mantle layer was removed from the compression coated tablet exposing the compressed core. The (axial×radial) dimensions of the compressed core were 3.66×6.62 mm. The axial mantle thickness was then calculated by taking half of the difference between the axial measurements of the compression coated tablet and the compressed core. The same was done for the radial mantle thickness calculation. The axial and radial mantle thicknesses were calculated to be 1.13 mm and 1.46 mm, respectively.

### Example 16

Compression coated tablets containing dimethyl fumarate were made having the ingredients shown in Table 15:

TABLE 15

| | | | Quantity | Quantity |
|---|---|---|---|---|
| Component | Manufacturer | Role | (mg/tablet) | (% w/w) |
| Dimethyl | TCI | Drug | 120.00 | 45.39 |
| Fumarate | (Portland, OR) | substance | | |
| Hydroxypropyl | Ashland | Binder | 3.63 | 1.37 |
| Cellulose | (Wilmington, DE) | | | |
| Hypromellose | Dow Chemical | Sustained | 9.41 | 3.56 |
| 2208 | (Midland, MI) | Release | | |
| (100000 mPa · s) | | Polymer | | |
| Silicon | Degussa | Glidant | 0.67 | 0.25 |
| Dioxide | (Parsippany, NJ) | | | |
| Magnesium | Mallinckrodt | Lubricant | 0.67 | 0.25 |
| Stearate | (St. Louis, MO) | | | |
| | | Total Core | 134.38 | 50.83 |
| Lactose | Foremost | Filler | 86.45 | 32.70 |
| Hydrate | (Rothschild, WI) | | | |
| Hypromellose | Dow Chemical | Sustained | 42.25 | 15.98 |
| 2208 | (Midland, MI) | Release | | |
| (100 mPa · s) | | Polymer | | |
| Magnesium | Mallinckrodt | Lubricant | 1.30 | 0.49 |
| Stearate | (St. Louis, MO) | | | |
| | | Total Mantle | 130.00 | 49.17 |
| | | Total Tablet | 264.38 | 100.00 |

The tablets were made according to the following steps. The core tablets were prepared using the same equipment, procedures, and material quantity as those described in Example 15.

The mantle blend was prepared using the same equipment and procedures as those described in Example 15, but with the following differences. The mantle blend was applied to the core tablets using the Carver Press with 5/16 inch (7.94 mm) round standard concave tooling. Half the mantle blend (65.0 mg) was weighed out, added to the die, and tamped slightly to flatten. Then, the core tablet was placed into the die and pressed down gently into the mantle blend. The second half of the mantle blend (65.0 mg) was then added on top of the core tablet and the mantle was compressed using 1.6 metric ton (MT) force. The final compression coated tablets had a total weight of 264.4 mg with a dimethyl fumarate loading of 120 mg (45.39%). The (axial x radial) dimensions of the compression coated tablet were 4.84×7.97 mm. The mantle layer was removed from the compression coated tablet exposing the compressed core. The (axial x radial) dimensions of the compressed core were 3.52×6.63 mm. The axial mantle thickness was then calculated by taking half of the difference between the axial measurements of the compression coated tablet and the compressed core. The same was done for the radial mantle thickness calculation. The axial and radial mantle thicknesses were calculated to be 0.66 mm and 0.67 mm, respectively.

### Example 17

Compression coated tablets containing dimethyl fumarate were made having the ingredients shown in Table 16:

TABLE 16

| | | | Quantity | Quantity |
|---|---|---|---|---|
| Component | Manufacturer | Role | (mg/tablet) | (% w/w) |
| Dimethyl | TCI | Drug | 120.00 | 28.96 |
| Fumarate | (Portland, OR) | substance | | |
| Hydroxypropyl | Ashland | Binder | 3.63 | 0.88 |
| Cellulose | (Wilmington, DE) | | | |
| Hypromellose | Dow Chemical | Sustained | 9.41 | 2.27 |
| 2208 | (Midland, MI) | Release | | |
| (100000 mPa · s) | | Polymer | | |
| Silicon Dioxide | Degussa | Glidant | 0.67 | 0.16 |
| | (Parsippany, NJ) | | | |
| Magnesium | Mallinckrodt | Lubricant | 0.67 | 0.16 |
| Stearate | (St. Louis, MO) | | | |
| | | Total Core | 134.38 | 32.43 |
| Lactose Hydrate | Foremost | Filler | 165.20 | 39.87 |
| | (Rothschild, WI) | | | |
| Hypromellose | Dow Chemical | Sustained | 112.00 | 27.03 |
| 2208 | (Midland, MI) | Release | | |
| (100 mPa · s) | | Polymer | | |
| Magnesium | Mallinckrodt | Lubricant | 2.80 | 0.68 |
| Stearate | (St. Louis, MO) | | | |
| | | Total Mantle | 280.00 | 67.57 |
| | | Total Tablet | 414.38 | 100.00 |

The tablets were made according to the following steps. The core tablets were prepared using the same equipment, procedures, and material quantity as those described in Example 15.

The mantle blend was prepared using a direct blending process and a batch size of 60 g. The hypromellose 2208 (100 MPa·s viscosity) and lactose hydrate were passed through a 600 micron mesh screen, combined in a glass jar and blended on a Turbula mixer for 5 minutes. Magnesium stearate was passed through a 600 micron screen and added to the blend and blended an additional 1.5 minutes. The mantle blend was then applied to the core tablets using the Carver Press with 9.50 mm round standard concave tooling. Half the mantle blend (140.0 mg) was weighed out, added to the die, and tamped slightly to flatten. Then, the core tablet was placed into the die and pressed down gently into the mantle blend. The second half of the mantle blend (140.0 mg) was then added on top of the core tablet and the mantle was compressed using 1.6 metric ton (MT) force. The final compression coated tablets had a total weight of 414.4 mg with a dimethyl fumarate loading of 120 mg (28.96%). The (axial x radial) dimensions of the compression coated tablet were 6.10×9.52 mm. The mantle layer was removed from the compression coated tablet exposing the compressed core. The (axial×radial) dimensions of the compressed core were 3.65×6.53 mm. The axial mantle thickness was then calculated by taking half of the difference between the axial measurements of the compression coated tablet and the compressed core. The same was done for the radial mantle thickness calculation. The axial and radial mantle thicknesses were calculated to be 1.23 mm and 1.50 mm, respectively.

### Example 18

Compression coated tablets containing dimethyl fumarate were made having the ingredients shown in Table 17:

TABLE 17

| | | | Quantity | Quantity |
|---|---|---|---|---|
| Component | Manufacturer | Role | (mg/tablet) | (% w/w) |
| Dimethyl | TCI | Drug | 120.00 | 45.39 |
| Fumarate | (Portland, OR) | substance | | |
| Hydroxypropyl | Ashland | Binder | 3.63 | 1.37 |
| Cellulose | (Wilmington, DE) | | | |
| Hypromellose | Dow Chemical | Sustained | 9.41 | 3.56 |
| 2208 | (Midland, MI) | Release | | |
| (100000 mPa · s) | | Polymer | | |
| Silicon Dioxide | Degussa | Glidant | 0.67 | 0.25 |
| | (Parsippany, NJ) | | | |
| Magnesium | Mallinckrodt | Lubricant | 0.67 | 0.25 |
| Stearate | (St. Louis, MO) | | | |
| | | Total Core | 134.38 | 50.83 |
| Lactose Hydrate | Foremost | Filler | 76.70 | 29.01 |
| | (Rothschild, WI) | | | |
| Hypromellose | Dow Chemical | Sustained | 52.00 | 19.67 |
| 2208 | (Midland, MI) | Release | | |
| (100 mPa · s) | | Polymer | | |
| Magnesium | Mallinckrodt | Lubricant | 1.30 | 0.49 |
| Stearate | (St. Louis, MO) | | | |
| | | Total Mantle | 130.00 | 49.17 |
| | | Total Tablet | 264.38 | 100.00 |

The tablets were made according to the following steps. The core tablets were prepared using the same equipment, procedures, and material quantity as those described in Example 15.

The mantle blend was prepared using the same equipment and procedures as those described in Example 17, but with the following differences. The mantle blend was applied to the core tablets using the Carver Press with 5/16 inch (7.94 mm) round standard concave tooling. Half the mantle blend (65.0 mg) was weighed out, added to the die, and tamped slightly to flatten. Then, the core tablet was placed into the die and pressed down gently into the mantle blend. The second half of the mantle blend (65.0 mg) was then added on top of the core tablet and the mantle was compressed using 1.6 metric ton (MT) force. The final compression coated tablets had a total weight of 264.4 mg with a dimethyl fumarate loading of 120 mg (45.39%). The (axial x radial) dimensions of the compression coated tablet were 4.84×7.95 mm. The mantle layer was removed from the compression coated tablet exposing the compressed core. The (axial x radial) dimensions of the compressed core were 3.42×6.67 mm. The axial mantle thickness was then calculated by taking half of the difference between the axial measurements of the compression coated tablet and the compressed core. The same was done for the radial mantle thickness calculation. The axial and radial mantle thicknesses were calculated to be 0.71 mm and 0.64 mm, respectively.

### Example 19

Compression coated tablets containing dimethyl fumarate were made having the ingredients shown in Table 18:

TABLE 18

| | | | Quantity | Quantity |
|---|---|---|---|---|
| Component | Manufacturer | Role | (mg/tablet) | (% w/w) |
| Dimethyl | TCI | Drug | 240.00 | 47.17 |
| Fumarate | (Portland, OR) | substance | | |
| Hydroxypropyl | Ashland | Binder | 7.26 | 1.43 |
| Cellulose | (Wilmington, DE) | | | |
| Hypromellose | Dow Chemical | Sustained | 18.81 | 3.70 |
| 2208 | (Midland, MI) | Release | | |
| (100000 mPa · s) | | Polymer | | |
| Silicon Dioxide | Degussa | Glidant | 1.34 | 0.26 |
| | (Parsippany, NJ) | | | |
| Magnesium | Mallinckrodt | Lubricant | 1.34 | 0.26 |
| Stearate | (St. Louis, MO) | | | |
| | | Total Core | 268.76 | 52.83 |
| Lactose Hydrate | Foremost | Filler | 141.60 | 27.83 |
| | (Rothschild, WI) | | | |
| Hypromellose | Dow Chemical | Sustained | 96.00 | 18.87 |
| 2208 | (Midland, MI) | Release | | |
| (100 mPa · s) | | Polymer | | |
| Magnesium | Mallinckrodt | Lubricant | 2.40 | 0.47 |
| Stearate | (St. Louis, MO) | | | |
| | | Total Mantle | 240.00 | 47.17 |
| | | Total Tablet | 508.76 | 100.00 |

The tablets were made according to the following steps. The core tablets were prepared using the same equipment and procedures as those described in Example 15, but with the following differences. Core tablets (268.8 mg) were compressed using a Carver Press with 5/16 inch (7.94 mm) round standard concave tooling at 1.0 metric ton (MT) force. The mantle blend was prepared using the same equipment and procedures as those described in Example 17, but with the following differences. The mantle blend was applied to the core tablets using the Carver Press with 13/32 inch (10.32 mm) round standard concave tooling. Half the mantle blend (120.0 mg) was weighed out, added to the die, and tamped slightly to flatten. Then, the core tablet was placed into the die and pressed down gently into the mantle blend. The second half of the mantle blend (120.0 mg) was then added on top of the core tablet and the mantle was compressed using 2.0 MT force. The final compression coated tablets had a total weight of 508.8 mg with a dimethyl fumarate loading of 240 mg (47.17%). The (axial×radial) dimensions of the compression coated tablet were 5.58×10.33 mm. The mantle layer was removed from the compression coated tablet exposing the compressed core. The (axial×radial) dimensions of the compressed core were 4.09×8.71 mm. The axial mantle thickness was then calculated by taking half of the difference between the axial measurements of the compression coated tablet and the compressed core. The same was done for the radial mantle thickness calculation. The axial and radial mantle thicknesses were calculated to be 0.75 mm and 0.81 mm, respectively.

### Example 20

Compression coated tablets containing (N,N-Diethylcarbamoyl)methyl methyl (2E)but-2-ene-1,4-dioate were made having the ingredients shown in Table 19:

TABLE 19

| | | | Quantity | Quantity |
|---|---|---|---|---|
| Component | Manufacturer | Role | (mg/tablet) | (% w/w) |
| (N,N- | XenoPort | Drug | 400.00 | 45.26 |
| Diethylcarbamoyl)methyl | (Santa Clara, CA) | substance | | |
| methyl (2E)but-2-ene- | | | | |
| 1,4- [symbol] | | | | |
| Hydroxypropyl Cellulose | Hercules | Binder | 12.37 | 1.40 |
| | (Wilmington, DE) | | | |
| Hypromellose 2208 | Dow Chemical | Sustained | 31.72 | 3.59 |
| (100000 mPa · s) | (Midland, MI) | Release | | |
| | | Polymer | | |
| Silicon Dioxide | Cabot | Glidant | 2.27 | 0.26 |
| | (Tuscola, IL) | | | |
| Magnesium Stearate | Mallinckrodt | Lubricant | 6.80 | 0.77 |
| | (St. Louis, MO) | | | |
| | | Total Core | 453.16 | 51.31 |
| Lactose Hydrate | Foremost | Filler | 253.70 | 28.73 |
| | (Rothschild, WI) | | | |
| Hypromellose 2208 | Dow Chemical | Sustained | 172.00 | 19.48 |
| (100 mPa · s) | (Midland, MI) | Release | | |
| | | Polymer | | |
| Magnesium Stearate | Mallinckrodt | Lubricant | 4.30 | 0.49 |
| | (St. Louis, MO) | | | |
| | | Total Mantle | 430.00 | 48.69 |
| | | Total Tablet | 883.16 | 100.00 |

[symbol]

indicates data missing or illegible when filed

The tablets were made according to the following steps. The core tablets were prepared using a wet granulation process. The granulation batch size was 170 g. (N,N-Diethylcarbamoyl)methyl methyl (2E)but-2-ene-1,4-dioate and hydroxypropyl cellulose were granulated with purified water using a Diosna P1/6 equipped with a 1 L bowl. The wet granules were passed through the Quadro Comil U5 with a 2769 micron screen at 3000 rpm and dried in a Glatt Fluid Bed Drier at 40° C. for 29 minutes. The dried granules and half of the silicon dioxide were combined in a 1 quart (0.95 L) V-blender and blended for 5 minutes, then passed through the Quadro Comil U5 with a 1270 micron screen at 3000 rpm. The milled blend was then blended for an additional 10 minutes.

The core blend batch size was 54.8 g. The second half of the silicon dioxide and hypromellose 2208 (100000 MPa·s viscosity) were then passed through a 600 micron mesh screen, combined with the blend in a glass jar and blended on a Turbula mixer for 5 minutes. Magnesium stearate was passed through a 600 micron screen and added to the blend before blending an additional 1.5 minutes. Core tablets (453.16 mg) were compressed using a Carver Press with 13/32 inch (10.32 mm) round standard concave tooling at 0.8 metric ton (MT) force. The mantle blend was prepared using the same equipment and procedures as those described in Example 17, but with the following differences. The mantle blend was applied to the core tablets using the Carver Press with 1/2inch (12.70 mm) round standard flat tooling. 190.0 mg of the mantle blend was weighed out, added to the die, and tamped slightly to flatten. Then, the core tablet was placed into the die and pressed down gently into the mantle blend. The remaining portion of the mantle blend (240.0 mg) was then added on top of the core tablet and the mantle was compressed using 1.6 MT force. The final compression coated tablets had a total weight of 883.16 mg with a (N,N-Diethylcarbamoyl)methyl methyl (2E)but-2-ene-1,4-dioate loading of 400 mg (45.26%). The (axial×radial) dimensions of the compression coated tablet were 5.61×12.74 mm. The mantle layer was removed from the compression coated tablet exposing the compressed core. The (axial×radial) dimensions of the compressed core were 4.09×11.26 mm. The axial mantle thickness was then calculated by taking half of the difference between the axial measurements of the compression coated tablet and the compressed core. The same was done for the radial mantle thickness calculation. The axial and radial mantle thicknesses were calculated to be 0.76 mm and 0.74 mm, respectively.

### Example 21

A two-stage dissolution method was used to determine the in vitro dissolution profile of dosage forms prepared according to Examples 15, 16, 17, and 18 in order to mimic the conditions of a dosage form as it transits the gastrointestinal tract. Thus, the dosage forms were first placed into a dissolution medium having a pH of 1.2, to mimic the conditions of the stomach, and then placed into a dissolution medium of pH 6.8, to mimic the conditions of the intestines. The dissolution vessel (USP, Type I, basket) initially contained 750 ml of 0.1 N hydrochloric acid (pH 1.2). After 2 hours of dissolution, 250 ml of 200 mM tribasic sodium phosphate was added to the vessel resulting in a pH adjustment from 1.2 to 6.8. The dissolution medium was kept at 37° C. and was agitated at 100 rpm.

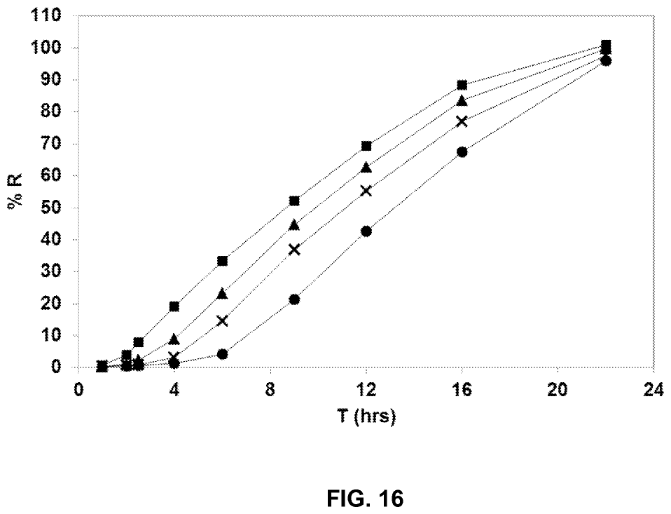

For the tested dosage forms, samples of the dissolution medium were withdrawn at the indicated time points shown in FIG. 16. The amount of dimethyl fumarate in the dissolution medium samples was determined by reverse phase HPLC using a C18 column and a 7 minute gradient method according to Table 4 where Mobile Phase A is water/0.1% H₃PO₄ and Mobile Phase B is water/acetonitrile/H₃PO₄ (10/90/0.1 by volume) with UV detection at 210 nm.

TABLE 20

| Time (minute) | % Mobile Phase A | % Mobile Phase B |
|---|---|---|
| 0 | 85 | 15 |
| 5 | 35 | 65 |
| 5.5 | 85 | 15 |
| 7 | 85 | 15 |

As shown in FIG. 16, for dosage forms prepared according to Example 15 (X-X symbols), drug release is delayed for approximately 2 hours, and thereafter the drug is released gradually, reaching more than 90% released at 20 hours. For dosage forms prepared according to Example 16 (▪-▪ symbols), drug release is delayed for approximately 1 hour, and thereafter the drug is released gradually, reaching more than 90% released at 17 hours. For dosage forms prepared according to Example 17 (●-● symbols), drug release is delayed for approximately 4 hours, and thereafter the drug is released gradually, reaching more than 90% released at 21 hours. For dosage forms prepared according to Example 18 (▴-▴ symbols), drug release is delayed for approximately 2 hours, and thereafter the drug is released gradually, reaching more than 90% released at 19 hours.

### Example 22

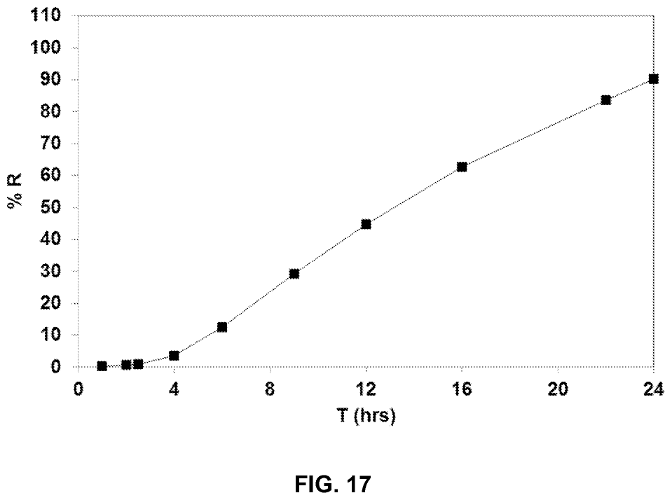

The dissolution profile from the compression coated tablets of Example 19 was tested according to the method described in Example 21. As shown in FIG. 17, for dosage forms prepared according to Example 19, drug release is delayed for approximately 2 hours, and thereafter the drug is released gradually, reaching more than 90% released at 24 hours.

### Example 23

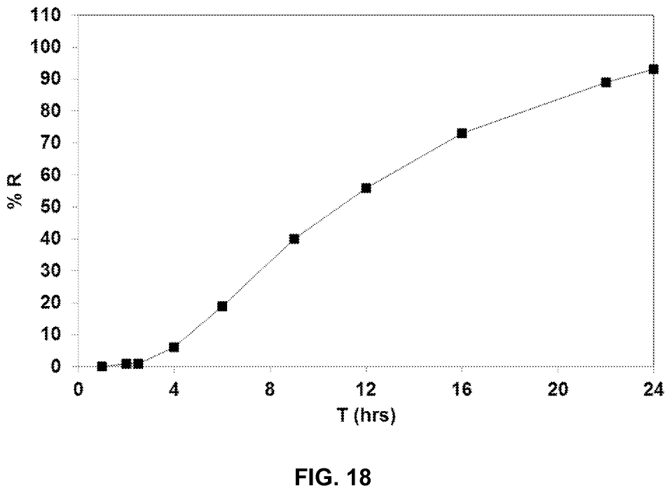

The dissolution profile from the compression coated tablets of Example 20 was tested according to the method described in Example 21, but for (N,N-Diethylcarbamoyl)methyl methyl (2E)but-2-ene-1,4-dioate. As shown in FIG. 18, for dosage forms prepared according to Example 20, drug release is delayed for approximately 2 hours, and thereafter the drug is released gradually, reaching more than 90% released at 23 hours.

### Example 24

On the surface of the punches of a tablet press of the type described in Manufacturing Example 1 of Ozeki et al., U.S. Pat. No. 7,811,488, the punches having a double structure with an inside diameter of 8.5 mm and an outside diameter of 10.0 mm and with a pressurizable flat edge, a small amount of magnesium stearate is added and as the lower central punch is kept in the lowered position, in the space above the lower central punch, enclosed by the lower outer punch, 15 mg of a 40:60 by weight mixture of lactose and hydroxypropylmethyl cellulose (HPMC) is added. Then the upper central punch and the lower central punch are moved towards each other and compression is applied manually causing the surface to become flat. Next, as the lower central punch is kept in the lowered position, in the space above the temporary moldings of lactose and HPMC, enclosed by the lower outer layer, 300 mg of a 90:10 by weight mixture of (N,N-Diethylcarbamoyl)methyl methyl (2E)but-2-ene-1,4-dioate and HPMC is added. Then the upper central punch and the lower central punch are moved towards each other and temporary compression is applied manually so as to maintain the molding shape. Next, as the bottom layer is kept in the lowered position, in the space in the die above and around the molding, made of lactose, HPMC and (N,N-Diethylcarbamoyl)methyl methyl (2E)but-2-ene-1,4-dioate, the remaining 60 mg of 40:60 by weight mixture of lactose and HPMC is added and the temporary (N,N-Diethylcarbamoyl)methyl methyl (2E)but-2-ene-1,4-dioate moldings are completely enclosed in lactose and HPMC. Then the upper central punch and the lower central punch are moved towards each other and, using a hydraulic hand press, the tablet is made with a compression force of about 1.4 ton. Each tablet weighs 375 mg, has a thickness of 3.40 mm, and contains 270 mg of (N, N-Diethylcarbamoyl)methyl methyl (2E)but-2-ene-1,4-dioate. The thickness, in the radial direction, of the outer lactose and HPMC mantle layer is 0.75 mm.

### Example 25

Tablets similar to those described in Example 24 are made using the same equipment and procedures, but with the following difference. For the tablet core, 300 mg of a 90:10 by weight mixture of dimethyl fumarate and HPMC is used. Each tablet weighs 375 mg, has a thickness of 3.40 mm, and contains 270 mg of dimethyl fumarate. The thickness of the outer lactose and HPMC mantle layer is 0.75 mm.

### Example 26

Compression coated tablets containing (N, N-Diethylcarbamoyl)methyl methyl (2E)but-2-ene-1.4-dioate were made having the ingredients shown in Table 21:

TABLE 21

| | | | Quantity | Quantity |
|---|---|---|---|---|
| Component | Manufacturer | Role | (mg/tablet) | (% w/w) |
| (N,N- | XenoPort | Drug | 400.00 | 51.72 |
| Diethylcarbamoyl)methyl | (Santa Clara, | substance | | |
| methyl (2E)but-2-ene- | CA) | | | |
| 1,4-dioate | | | | |
| Hydroxypropyl Cellulose | Hercules | Binder | 12.37 | 1.60 |
| | (Wilmington, DE) | | | |
| Hypromellose 2208 | Dow Chemical | Sustained | 26.60 | 3.44 |
| (100000 mPa · s) | (Midland, MI) | Release | | |
| | | Polymer | | |
| Silicon Dioxide | Cabot | Glidant | 2.22 | 0.29 |
| | (Tuscola, IL) | | | |
| Magnesium Stearate | Mallinckrodt | Lubricant | 2.22 | 0.29 |
| | (St. Louis, MO) | | | |
| | | Total Core | 443.41 | 57.33 |
| Lactose Hydrate | Foremost | Filler | 194.70 | 25.17 |
| | (Rothschild, WI) | | | |
| Hypromellose 2208 | Dow Chemical | Sustained | 132.00 | 17.07 |
| (100 mPa · s) | (Midland, MI) | Release | | |
| | | Polymer | | |
| Magnesium Stearate | Mallinckrodt | Lubricant | 3.30 | 0.43 |
| | (St. Louis, MO) | | | |
| | | Total Mantle | 330.00 | 42.67 |
| | | Total Tablet | 773.41 | 100.00 |

The tablets were made according to the following steps. The core tablets were prepared using a wet granulation process. The granulation batch size was 170 g. (N,N-Diethylcarbamoyl)methyl methyl (2E)but-2-ene-1,4-dioate and hydroxypropyl cellulose were granulated with purified water using a Diosna P1/6 equipped with a 1 L bowl. The wet granules were passed through the Quadro Comil U5 with a 2769 micron screen at 3000 rpm and dried in a Glatt Fluid Bed Drier at 40° C. for 29 minutes. The dried granules and half of the silicon dioxide were combined in a 1 quart (0.95 L) V-blender and blended for 5 minutes, then passed through the Quadro Comil U5 with a 1270 micron screen at 3000 rpm. The milled blend was then blended for an additional 10 minutes. The core blend batch size was 53.5 g. The second half of the silicon dioxide was then passed through a 850 micron mesh screen, combined with the blend in a glass jar and blended on a Turbula mixer for 3 minutes. Hypromellose 2208 (100000 MPa·s viscosity) was then passed through a 600 micron screen and blended for 5 minutes. Magnesium stearate was passed through a 600 micron screen and added to the blend before blending an additional 1.5 minutes. Core tablets (443.4 mg) were compressed using a Carver Press with 0.2746×0.5930 inch (6.97×15.06 mm) modified oval standard concave tooling at 0.7 metric ton (MT) force.

The mantle blend was prepared using a direct blending process and a batch size of 60 g. The hypromellose 2208 (100 MPa·s viscosity) and lactose hydrate were passed through a 600 micron mesh screen, combined in a glass jar and blended on a Turbula mixer for 5 minutes. Magnesium stearate was passed through a 600 micron screen and added to the blend and blended an additional 1.5 minutes. The mantle blend was applied to the core tablets using the Carver Press with 0.3531×0.6717 inch (8.97×17.06 mm) modified oval standard concave tooling. Half the mantle blend (165.0 mg) was weighed out, added to the die, and tamped slightly to flatten. Then, the core tablet was placed into the die and pressed down gently into the mantle blend. The second half of the mantle blend (165.0 mg) was then added on top of the core tablet and the mantle was compressed using 1.6 MT force. The final compression coated tablets had a total weight of 773.4 mg with a (N,N-Diethylcarbamoyl)methyl methyl (2E)but-2-ene-1,4-dioate loading of 400 mg (51.72%). The (axial×minor×major) dimensions of the compression coated tablet were 6.47×9.00×17.11 mm. The mantle layer was removed from the compression coated tablet exposing the compressed core. The (axial×minor×major) dimensions of the compressed core were 4.85×7.35×15.03 mm. The axial mantle thickness was then calculated by taking half of the difference between the axial measurements of the compression coated tablet and the compressed core. The same was done for the minor and major mantle thickness calculations. The axial, minor, and major mantle thicknesses were calculated to be 0.81 mm, 0.83 mm, and 0.79 mm, respectively.

### Example 27

Compression coated tablets containing (N,N-Diethylcarbamoyl)methyl methyl (2E)but-2-ene-1,4-dioate were made having the ingredients shown in Table 22:

TABLE 22

| | | | Quantity | Quantity |
|---|---|---|---|---|
| Component | Manufacturer | Role | (mg/tablet) | (% w/w) |
| (N,N- | XenoPort | Drug | 400.00 | 53.81 |
| Diethylcarbamoyl)methyl | (Santa Clara, CA) | substance | | |
| methyl (2E)but-2-ene-1,4- | | | | |
| dioate | | | | |
| Hydroxypropyl Cellulose | Hercules | Binder | 12.37 | 1.66 |
| | (Wilmington, DE) | | | |
| Hypromellose 2208 | Dow Chemical | Sustained | 26.60 | 3.58 |
| (100000 mPa · s) | (Midland, MI) | Release | | |
| | | Polymer | | |
| Silicon Dioxide | Cabot (Tuscola, | Glidant | 2.22 | 0.30 |
| | IL) | | | |
| Magnesium Stearate | Mallinckrodt | Lubricant | 2.22 | 0.30 |
| | (St. Louis, MO) | | | |
| | | Total Core | 443.41 | 59.65 |
| Lactose Hydrate | Foremost | Filler | 177.00 | 23.81 |
| | (Rothschild, WI) | | | |
| Hypromellose 2208 | Dow Chemical | Sustained | 120.00 | 16.14 |
| (100 mPa · s) | (Midland, MI) | Release | | |
| | | Polymer | | |
| Magnesium Stearate | Mallinckrodt | Lubricant | 3.00 | 0.40 |
| | (St. Louis, MO) | | | |
| | | Total Mantle | 300.00 | 40.35 |
| | | Total Tablet | 743.41 | 100.00 |

The tablets were made according to the following steps. The core tablets were prepared using the same equipment and procedures as those described in Example 26, but with the following differences.

Core tablets (443.4 mg) were compressed using a Carver Press with 0.2854×0.5709 inch (7.25×14.50 mm) oval standard concave tooling at 0.7 metric ton (MT) force.

The mantle blend was prepared using the same equipment and procedures as those described in Example 26, but with the following differences. The mantle blend was applied to the core tablets using the Carver Press with 0.3642×0.6496 inch (9.25×16.50 mm) oval standard concave tooling. Half the mantle blend (150.0 mg) was weighed out, added to the die, and tamped slightly to flatten. Then, the core tablet was placed into the die and pressed down gently into the mantle blend. The second half of the mantle blend (150.0 mg) was then added on top of the core tablet and the mantle was compressed using 1.6 MT force. The final compression coated tablets had a total weight of 743.4 mg with a (N,N-Diethylcarbamoyl)methyl methyl (2E)but-2-ene-1,4-dioate loading of 400 mg (53.81%). The (axial×minor×major) dimensions of the compression coated tablet were 6.26×9.27×16.54 mm. The mantle layer was removed from the compression coated tablet exposing the compressed core. The (axial×minor×major) dimensions of the compressed core were 4.76 ×7.87×15.22 mm. The axial mantle thickness was then calculated by taking half of the difference between the axial measurements of the compression coated tablet and the compressed core. The same was done for the minor and major mantle thickness calculations. The axial, minor, and major mantle thicknesses were calculated to be 0.75 mm, 0.70 mm, and 0.66 mm, respectively.

### Example 28

A two-stage dissolution method was used to determine the in vitro dissolution profile of dosage forms prepared according to Example 26 in order to mimic the conditions of a dosage form as it transits the gastrointestinal tract. Thus, the dosage forms were first placed into a dissolution medium having a pH of 1.2, to mimic the conditions of the stomach, and then placed into a dissolution medium of pH 6.8, to mimic the conditions of the intestines. The dissolution vessel (USP, Type I, basket) initially contained 750 ml of 0.1 N hydrochloric acid (pH 1.2). After 2 hours of dissolution, 250 mL of 200 mM tribasic sodium phosphate was added to the vessel resulting in a pH adjustment from 1.2 to 6.8. The dissolution medium was kept at 37° C. and was agitated at 100 rpm. For the tested dosage forms, samples of the dissolution medium were withdrawn at the indicated time points shown in the respective figures. The amount of (N,N-Diethylcarbamoyl)methyl methyl (2E)but-2-ene-1,4-dioate in the dissolution medium samples was determined by reverse phase HPLC using a C18 column and a 7 minute gradient method according to Table 23 where Mobile Phase A is water/0.1% H₃PO₄ and Mobile Phase B is water/acetonitrile/H₃PO₄ (10/90/0.1 by volume) with UV detection at 210 nm.

TABLE 23

| Time (minute) | % Mobile Phase A | % Mobile Phase B |
|---|---|---|
| 0 | 85 | 15 |
| 5 | 35 | 65 |
| 5.5 | 85 | 15 |
| 7 | 85 | 15 |

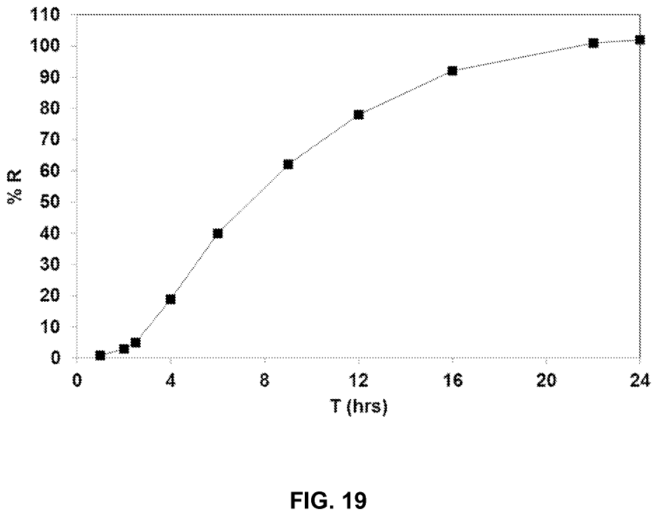

As shown in FIG. 19, for dosage forms prepared according to Example 26, drug release is delayed for approximately 1 hour, and thereafter the drug is released gradually, reaching more than 90% released at 16 hours.

### Example 29

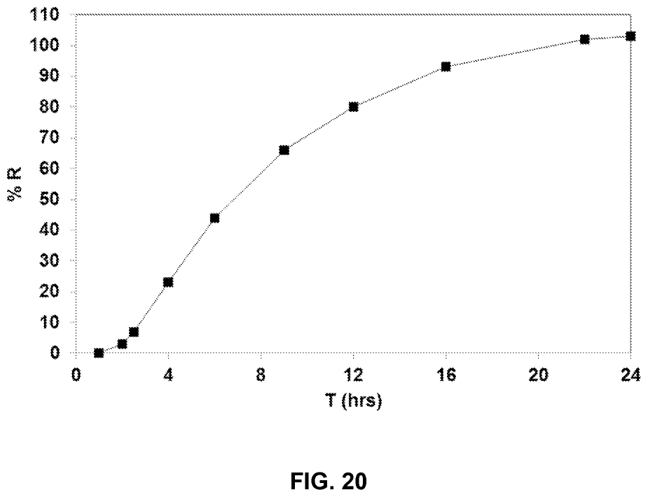

The dissolution profile from the compression coated tablets of Example 27 was tested according to the method described in Example 28. As shown in FIG. 20, for dosage forms prepared according to Example 27, drug release is delayed for approximately 1 hour, and thereafter the drug is released gradually, reaching more than 90% released at 16 hours.

### Example 30

Compression coated tablets containing N,N-Diethylcarbamoyl)methyl methyl (2E)but-2-ene-1,4-dioate were made having the ingredients shown in Table 24:

TABLE 24

| | | Quantity | Quantity | Quantity | Quantity |
|---|---|---|---|---|---|
| Component | Function | mg/tablet | (% w/w) | mg/tablet | (% w/w) |
| (N,N- | Drug | 100 | 24.5 | 200 | 34.4 |
| Diethylcarbamoyl)methyl | Substance | | | | |
| methyl (2E)but-2-ene- | | | | | |
| 1,4-dioate | | | | | |
| Hydroxypropyl Methyl | Granulation | 2.04 | 0.5 | 4.08 | 0.7 |
| Cellulose (Hypromellose | Binder | | | | |
| 2910 Methocel E5 LV) 5 | | | | | |
| mPas | | | | | |
| Hypromellose 2208 | Sustained | 14.75 | 3.6 | 15.53 | 2.7 |
| (Methocel K15 MCR) | Release | | | | |
| 15000 mPas | Agent | | | | |
| Colloidal Silicon di oxide | Glidant | 0.59 | 0.1 | 1.11 | 0.2 |
| (Cab-o-sil M5P/Aerosil | | | | | |
| 200 Pharma) | | | | | |
| Magnesium Stearate | Lubricant | 0.59 | 0.1 | 1.11 | 0.2 |
| (HyQual VG, Code 2257) | | | | | |
| Total Core | | 118 | 28.9 | 221.8 | 38.2 |
| Lactose Monohydrate | Diluent | 198.85 | 47.3 | 239.4 | 41.1 |
| Spray Dried (Foremost | | | | | |
| 316 Fast Flo) | | | | | |
| Hypromellose 2208 | Sustained | 94.25 | 23.1 | 117 | 20.1 |
| (Methocel K 100 LVCR) | Release | | | | |
| (100 mPa · s) | Agent | | | | |
| Magnesium Stearate | Lubricant | 2.9 | 0.7 | 3.6 | 0.6 |
| (HyQual VG, Code 2257) | | | | | |
| FD&C Blue 1 Aluminum | | | | | |
| lake | | | | | |

| | | | | |
|---|---|---|---|---|
| Total Mantle | 290 | 71.1 | 360 | 61.8 |
| Final Tablet Weight | 408 | 100 | 581.8 | 100 |

### Example 31

A randomized, double-blind, placebo-controlled, multicenter, 24-week study to assess the efficacy and safety of PPC-06 ((N,N-Diethylcarbamoyl)methyl methyl (2E)but-2-ene-1,4-dioate) extended release tablets according to Example 30 in subjects with moderate-to-severe plaque psoriasis was conducted.

### Methods:

Study subjects were enrolled at 76 US sites. Patients had chronic plaque psoriasis for more than 6 months, PASI (Psoriasis Area and Severity Index) scores ≥12, IGA (Investigator's Global Assessment) Scores ≥3, and psoriasis BSA (Body Surface Area) ≥10%. Altogether 426 subjects were randomized in a 1:1:1:1 ratio into 4 dose arms: 400 mg QD, 400 mg BID, 600 mg BID, and placebo. A 5-week titration phase was followed by 19 weeks of treatment. The co-primary end points at week 24 were the percentages of patients achieving an at least 75% reduction of their baseline PASI (“PASI-75”) and an IGA score of ‘clear’ or ‘almost clear’ (i.e., 0 or 1).

### Results

The primary efficacy analysis (FAS population) included 406 subjects: 400 mg QD (n=101), 400 mg BID (n=102), 600 mg BID (n=101) and placebo (n=102). At week 24 analysis, PASI-75 was achieved by 44.3%, 47.2% and 39.7% patients in PPC-06 600 mg BID, 400 mg BID and 400 mg QD treatment groups, respectively, compared to 20% in the placebo group (p=0.004, 0.002 and 0.004 for the PPC-06 600 mg BID, 400 mg BID and 400 mg QD treatment groups, respectively). Additionally, 44.4%, 41.4% and 35.7% of patients in the PPC-06 600 mg BID and 400 mg BID and 400 mg QD groups, respectively, achieved an IGA score of 0 or 1 at week 24, compared to 22% of patients in the placebo group (p=0.010, 0.021 and 0.044 for the PPC-06 600 mg BID, 400 mg BID and 400 mg QD treatment groups, respectively). Multiple Imputation was done for the missing efficacy data. Diarrhea was the most common Treatment-Emergent Adverse Event (TEAE), reported in 7%, 18%, 23% and 5% of patients in the PPC-06 400 mg QD, 400 mg BID, 600 mg BID, and placebo groups, respectively. Most cases of diarrhea were mild to moderate in severity. Nausea and abdominal pain were reported in less than 10% of the overall PPC-06 population. Flushing was reported in 1-2% of patients in the PPC-06 treatment arms and not at all in placebo arm. Two subjects developed CTCAE grade 3 lymphopenia during treatment with PPC-06, but recovered uneventfully on discontinuation of study drug. No new or unexpected adverse events related to PPC-06 were reported compared to what is known for DMF drugs. PPC-06 demonstrated significant efficacy compared to placebo over 24 weeks of treatment for three dose strengths.

Finally, it should be noted that there are alternative ways of implementing the embodiments provided by the present disclosure. Accordingly, the present embodiments are to be considered as illustrative and not restrictive, and the present disclosure is not to be limited to the details given herein, but may be modified within the scope and equivalents of the claim(s) issuing from a patent claiming priority hereto.

1-13. (canceled)

14. A method of treating psoriasis comprising orally administering a tablet to a subject in need thereof, wherein the tablet comprises:
(A) a core comprising (N,N-diethylcarbamoyl)methyl methyl (2E)but-2-ene-1,4-dioate in a dose of about 100 mg or about 200 mg; and
(B) a compressed coating layer surrounding the tablet core, wherein the compressed coating layer
(i) comprises hydroxypropyl methyl cellulose; and
(ii) is substantially free of ionizable polymer having carboxylic acid moieties;
wherein (N,N-diethylcarbamoyl)methyl methyl (2E)but-2-ene-1,4-dioate is the sole active ingredient in the tablet;
wherein the tablet, when subjected to an aqueous solution free of (N,N-diethylcarbamoyl)methyl methyl (2E)but-2-ene-1,4-dioate, releases at most 20% of the (N,N-diethylcarbamoyl)methyl methyl (2E)but-2-ene-1,4-dioate from the core over a period of 2 hours.

15. The method of claim 14, wherein the tablet further comprises binders, glidants, lubricants, diluents, or any combination thereof.

16. The method of claim 14, wherein the tablet has a compression coating weight of about 40% to about 75% of the total tablet weight.

17. The method of claim 14, wherein the tablet has a core weight to coating weight ratio of 1:1 to 1:3.

18. The method of claim 14, wherein the compressed coating layer at least 0.5 millimeters (mm).

19. The method of claim 14, wherein the tablet, when subjected to an aqueous solution free of (N,N-diethylcarbamoyl)methyl methyl (2E)but-2-ene-1,4-dioate, releases at least 80% of the (N,N-diethylcarbamoyl)methyl methyl (2E)but-2-ene-1,4-dioate over a period of at most 6 hours.

20. The method of claim 14, wherein the tablet results in a therapeutic concentration (N,N-diethylcarbamoyl)methyl methyl (2E)but-2-ene-1,4-dioate in blood plasma of at least 0.7 μg/ml at a time 24 hours after administration to the subject.

21. The method of claim 14, wherein the tablet results in an area under a concentration of (N,N-diethylcarbamoyl)methyl methyl (2E)but-2-ene-1,4-dioate in blood plasma versus time curve (AUC) of at least 12.0 μg·hr/ml over 24 hours after administration to the subject.

22. The method of claim 14, wherein the method occurs for a period of at least four months.

23. The method of claim 14, wherein tablet is administered once daily, twice daily, three times daily, or four times daily.

24. The method of claim 23, wherein about 400 mg (N,N-diethylcarbamoyl)methyl methyl (2E)but-2-ene-1,4-dioate is administered daily.

25. The method of claim 23, wherein about 400 mg (N,N-diethylcarbamoyl)methyl methyl (2E)but-2-ene-1,4-dioate is administered daily.

26. The method of claim 23, wherein about 600 mg (N,N-diethylcarbamoyl)methyl methyl (2E)but-2-ene-1,4-dioate is administered daily.

27. The method of claim 14, wherein the method provides at least 39.7% of patients at least 75% reduction in the baseline PASI score (PASI-75).

28. The method of claim 14, wherein method provides a reduction in the Investigator's Global Assessment (IGA) score.

29. The method of claim 28, wherein the method provides at least 35.7% of patients an IGA score reduced to 0 or 1.

30. The method of claim 14, wherein the psoriasis is moderate to severe plaque psoriasis.

31. The method of claim 14, wherein the viscosity of the hydroxypropyl methyl cellulose is about 100 mPa·s.

32. The method of claim 14, wherein the tablet, when subjected to an in vitro dissolution test employing a dissolution medium of 750 mL of 0.1N hydrochloric acid, at pH 1.2, for a period of 2 hours, followed by addition of 250 mL of 200 mM tribasic sodium phosphate buffer resulting in the dissolution medium pH adjustment to 6.8, the dissolution medium being maintained at 37° C. and stirred at 100 rpm, releases at most 90 wt % of the (N,N-diethylcarbamoyl)methyl methyl (2E)but-2-ene-1,4-dioate over a period of at least about 8 hours after the start of the in vitro dissolution test.
